SEQUENCE LISTING (12 sequences)

SEQ ID NO: 1 - DNA, 1332 bp, Serratia symbiotica
  source 1..1332: mol_type = genomic DNA
atgaaatacctgctgccgaccgctgctgctggtctgctgctcctcgctgcccagccggcgatggccatgtctgttatcgcggttctggaatccaacctggctggcaacggcacgctggttatccaggcggctaaactgaaaggttacaagaccttgttcgtttgcggtaacaaagcggaatacgctaacaccgttatcaatccgatcgatttcgctgacgaagttgcggttgttgacagctacgacatcgcgaaactgctggcgttcttccagagctatccagataccgtagacgcggttatggcgttcgatgacttccgtatgatccaggcagcactgatcaaccagttcttgaacctgccatacgctccagctgttgaagcgctgctgaccgttcgtttcaaacacttgctgcgtgagaaactgaacaacacggcgtacgcgatcgacttcactcgtctggcgggtgactccaccgctcaccaggcgctgctgaaatatccgtgcgttatcaaaccggttgatgaatctggttctatcggtgttaaagtttgccactccaaatctgaatctgacgaagctatcgactacattctgtctctgccgaaatacaacggccgtggtttcactgtttccaaagacatcttggtggaagaatttatcactggtgaagaatactccgcggaactggtttgggattgcgagaacgaagattggcgtctgctgggtgtaactcagaaattcgtgactccgccgccattctgcgttgagaaagcgcacatctttccatacgcggacggttctgagttctccgaacaggttagcctgcacgcaaaccgtatcttgaaacacatcggtctgcgtaacacctttctgcacatggagttcaaattctccgacggtcgtttcgatgttatcgaaatcaacccgcgtctgggtggcgatatgatcatcgaactgatgaagaacgcgaaaggttacgacgcagctggtctgatgctggaagcgaacatcaaccagccgctgagcatcgagaacaccggtgttcagggtgatgcgtctgcgatcgtttacatcaccgacagccgttgcggtcacatcaccgatattaccgttgaatccgatggcgacatcttcagccgtatcaacatcttctctctgccaaagactctgaaaggcctgcgttcttctgaagatcgtctgggttacgttatcttgaacaaagatcgttaccagcagatcgaactgcgtgttaacgctttgctggatggtaacggtttcaacatcactcagagcgcgctgtctctcgagcaccaccaccaccaccactga

SEQ ID NO: 2 - DNA, 1491 bp, Ralstonia pseudosolanacearum
  source 1..1491: mol_type = genomic DNA
atggtagtccgtcgtaccgtgcttaaggcgattgcgggtacctctgttgccaccgtcttcgcgggaaagttaaccggcctgtctgctgtcgcagctgacgcagctccgctgcgtgtccgccgtaatcttcatggaatgaaaatggatgatcctgatctgagcgcgtatcgggagtttgtcggtatcatgaaaggtaaagatcagacccaggcgcttagctggttaggctttgccaatcagcatggcacgctgaatggcggatacaaatattgcccgcatggcgactggtattttctgccctggcatcgtggctttgtgctgatgtatgaacgtgctgtggcggcgttaacaggctataaaacctttgctatgccatattggaattggaccgaagatcgtctgttaccggaagcgtttactgcgaaaacctataatggcaagaccaatccgctgtacgtcccaaaccgtaatgaacttaccggcccatatgcactgactgacgcgatcgtcggccaaaaagaagtgatggataaaatctatgcggaaaccaactttgaagtatttggaacaagccggagcgtggatcggagtgtgcgtcccccactggtgcaaaatagcttagatccgaaatgggtgccgatgggcggtggaaatcagggcattctggaacgtaccccgcataacacagtgcacaataacattggtgcctttatgccaacagccgcctccccgcgtgatcccgtatttatgatgcaccacggcaacattgaccgtgtgtgggcaacctggaatgctctgggccgtaagaatagcacggatcctttgtggctgggcatgaagtttcctaataactacatcgacccccagggccgttactatacccagggtgtgagcgacctgctgagcaccgaggccttgggctatcgttacgatgtgatgcctcgtgcagataataaggtcgtcaataatgcgcgtgcggaacatctgctggcgctgtttaaaaccggggatagcgtgaaactggcagatcatatacgtttgcgtagcgttcttaagggcgagcatcccgttgcgacggctgtggaacccttgaatagcgcggtgcagtttgaagcgggcacggtgactggggcgttaggcgcggatgtggggaccggctcaaccaccgaagttgtggcactgatcaagaatatacgcataccgtataatgtgattagcattcgtgtatttgttaatctgccgaatgcgaatctggatgtgccagaaacagatccgcatttcgtgacatcgctgagctttctgacccatgcagctggacatgaccaccatgcgctgccgagcacgatggtaaatctgaccgataccctgaaagccctgaacattcgggatgacaatttttctataaatcttgtagcggtgccgcagcctggcgtggcggttgaatcgagcggaggcgtgaccccggaaagcattgaggtggcagtgatttaa

SEQ ID NO: 3 - DNA, 1266 bp, Serratia symbiotica
  source 1..1266: mol_type = genomic DNA
atgtctgttatcgcggttctggaatccaacctggctggcaacggcacgctggttatccaggcggctaaactgaaaggttacaagaccttgttcgtttgcggtaacaaagcggaatacgctaacaccgttatcaatccgatcgatttcgctgacgaagttgcggttgttgacagctacgacatcgcgaaactgctggcgttcttccagagctatccagataccgtagacgcggttatggcgttcgatgacttccgtatgatccaggcagcactgatcaaccagttcttgaacctgccatacgctccagctgttgaagcgctgctgaccgttcgtttcaaacacttgctgcgtgagaaactgaacaacacggcgtacgcgatcgacttcactcgtctggcgggtgactccaccgctcaccaggcgctgctgaaatatccgtgcgttatcaaaccggttgatgaatctggttctatcggtgttaaagtttgccactccaaatctgaatctgacgaagctatcgactacattctgtctctgccgaaatacaacggccgtggtttcactgtttccaaagacatcttggtggaagaatttatcactggtgaagaatactccgcggaactggtttgggattgcgagaacgaagattggcgtctgctgggtgtaactcagaaattcgtgactccgccgccattctgcgttgagaaagcgcacatctttccatacgcggacggttctgagttctccgaacaggttagcctgcacgcaaaccgtatcttgaaacacatcggtctgcgtaacacctttctgcacatggagttcaaattctccgacggtcgtttcgatgttatcgaaatcaacccgcgtctgggtggcgatatgatcatcgaactgatgaagaacgcgaaaggttacgacgcagctggtctgatgctggaagcgaacatcaaccagccgctgagcatcgagaacaccggtgttcagggtgatgcgtctgcgatcgtttacatcaccgacagccgttgcggtcacatcaccgatattaccgttgaatccgatggcgacatcttcagccgtatcaacatcttctctctgccaaagactctgaaaggcctgcgttcttctgaagatcgtctgggttacgttatcttgaacaaagatcgttaccagcagatcgaactgcgtgttaacgctttgctggatggtaacggtttcaacatcactcagagcgcgctgtctctcgagcaccaccaccaccaccactga

SEQ ID NO: 4 - protein, 443 aa, Serratia symbiotica
MKYLLPTAAAGLLLLAAQPAMAMSVIAVLESNLAGNGTLVIQAAKLKGYKTLFVCGNKAEYANTVINPIDFADEVAVVDSYDIAKLLAFFQSYPDTVDAVMAFDDFRMIQAALINQFLNLPYAPAVEALLTVRFKHLLREKLNNTAYAIDFTRLAGDSTAHQALLKYPCVIKPVDESGSIGVKVCHSKSESDEAIDYILSLPKYNGRGFTVSKDILVEEFITGEEYSAELVWDCENEDWRLLGVTQKFVTPPPFCVEKAHIFPYADGSEFSEQVSLHANRILKHIGLRNTFLHMEFKFSDGRFDVIEINPRLGGDMIIELMKNAKGYDAAGLMLEANINQPLSIENTGVQGDASAIVYITDSRCGHITDITVESDGDIFSRINIFSLPKTLKGLRSSEDRLGYVILNKDRYQQIELRVNALLDGNGFNITQSALSLEHHHHHH

SEQ ID NO: 5 - protein, 496 aa, Ralstonia pseudosolanacearum
MVVRRTVLKAIAGTSVATVFAGKLTGLSAVAADAAPLRVRRNLHGMKMDDPDLSAYREFVGIMKGKDQTQALSWLGFANQHGTLNGGYKYCPHGDWYFLPWHRGFVLMYERAVAALTGYKTFAMPYWNWTEDRLLPEAFTAKTYNGKTNPLYVPNRNELTGPYALTDAIVGQKEVMDKIYAETNFEVFGTSRSVDRSVRPPLVQNSLDPKWVPMGGGNQGILERTPHNTVHNNIGAFMPTAASPRDPVFMMHHGNIDRVWATWNALGRKNSTDPLWLGMKFPNNYIDPQGRYYTQGVSDLLSTEALGYRYDVMPRADNKVVNNARAEHLLALFKTGDSVKLADHIRLRSVLKGEHPVATAVEPLNSAVQFEAGTVTGALGADVGTGSTTEVVALIKNIRIPYNVISIRVFVNLPNANLDVPETDPHFVTSLSFLTHAAGHDHHALPSTMVNLTDTLKALNIRDDNFSINLVAVPQPGVAVESSGGVTPESIEVAVI

SEQ ID NO: 6 - protein, 421 aa, Serratia symbiotica
MSVIAVLESNLAGNGTLVIQAAKLKGYKTLFVCGNKAEYANTVINPIDFADEVAVVDSYDIAKLLAFFQSYPDTVDAVMAFDDFRMIQAALINQFLNLPYAPAVEALLTVRFKHLLREKLNNTAYAIDFTRLAGDSTAHQALLKYPCVIKPVDESGSIGVKVCHSKSESDEAIDYILSLPKYNGRGFTVSKDILVEEFITGEEYSAELVWDCENEDWRLLGVTQKFVTPPPFCVEKAHIFPYADGSEFSEQVSLHANRILKHIGLRNTFLHMEFKFSDGRFDVIEINPRLGGDMIIELMKNAKGYDAAGLMLEANINQPLSIENTGVQGDASAIVYITDSRCGHITDITVESDGDIFSRINIFSLPKTLKGLRSSEDRLGYVILNKDRYQQIELRVNALLDGNGFNITQSALSLEHHHHHH

SEQ ID NO: 7 - DNA, 60 bp, synthetic construct
  source 1..60: mol_type = other DNA
tgctggtctgctgctcctcgctgcccagccggcgatggccatgtctgttatcgcggttct

SEQ ID NO: 8 - DNA, 60 bp, synthetic construct
  source 1..60: mol_type = other DNA
ctttgttagcagccggatctcagtggtggtggtggtggtgctcgagagacagcgcgctct

SEQ ID NO: 9 - DNA, 60 bp, synthetic construct
  source 1..60: mol_type = other DNA
tagaaataattttgtttaactttaagaaggagatatacatatggtagtccgtcgtaccgt

SEQ ID NO: 10 - DNA, 60 bp, synthetic construct
  source 1..60: mol_type = other DNA
ccaactcagcttcctttcgggctttgttagcagccggatcttaaatcactgccacctcaa

SEQ ID NO: 11 - DNA, 60 bp, synthetic construct
  source 1..60: mol_type = other DNA
tagaaataattttgtttaactttaagaaggagatatacatatgtctgttatcgcggttct

SEQ ID NO: 12 - DNA, 60 bp, synthetic construct; identical to SEQ ID NO: 8
  source 1..60: mol_type = other DNA

(19) United States
(12) Patent Application Publication
(10) Pub. No.: US 2026/0185133 A1
(43) Pub. Date: Jul. 2, 2026

(54) GENETICALLY MODIFIED MICROORGANISM FOR ENHANCING MELANIN PRODUCTION EFFICIENCY AND METHOD FOR PRODUCING MELANIN

(71) Applicant: INDUSTRIAL TECHNOLOGY RESEARCH INSTITUTE, Hsinchu (TW)

(72) Inventors: Jhong-De LIN, Hsinchu City (TW); Hung-Yu LIAO, Taoyuan City (TW); Hsiang-Yuan CHU, Hsinchu City (TW)

(73) Assignee: INDUSTRIAL TECHNOLOGY RESEARCH INSTITUTE, Hsinchu (TW)

(21) Appl. No.: 19/427,106

(22) Filed: Dec. 19, 2025

(30) Foreign Application Priority Data
Dec. 30, 2024 (TW) 113151480

(51) Int. Cl. C12P 17/10; C12N 9/00; C12N 9/02; C12N 15/70

(52) U.S. Cl. CPC C12P 17/10; C12N 9/0071; C12N 9/93; C12N 15/70; C12Y 114/18001; C12Y 603/02013; C12N 2800/101

(57) ABSTRACT

A genetically modified microorganism for enhancing melanin production efficiency is provided. The genetically modified microorganism for enhancing melanin production efficiency includes a first exogenous nucleic acid and a second exogenous nucleic acid. The first exogenous nucleic acid includes a nucleic acid encoding a secreted L-amino acid ligase, wherein secreted L-amino acid ligase is capable of linking two amino acids to form a dipeptide. The second exogenous nucleic acid includes a nucleic acid encoding tyrosinase.

### CROSS REFERENCE TO RELATED APPLICATIONS

This application claims priority of Taiwan Patent Application No. 113151480, filed on Dec. 30, 2024, the entirety of which is incorporated by reference herein.

### REFERENCE TO AN ELECTRONIC SEQUENCE LISTING

The contents of the electronic sequence listing (9044C-P240196300-US_ST26_Seq_Listing.xml; Size: 18,614 bytes; and Date of Creation: Apr. 9, 2025) is herein incorporated by reference in its entirety.

### TECHNICAL FIELD

The present invention relates to a genetically modified microorganism and a method for using it, and in particular, it relates to a genetically modified microorganism for enhancing melanin production efficiency and a method for producing melanin.

### BACKGROUND

Biosynthesis has become one of the main production methods for producing various chemicals today. How to further increase the production of biosynthetic chemicals is a major issue. Moreover, how to effectively produce target chemicals is one of the main core issues of biosynthetic chemicals.

The biosynthesis of chemicals is performed by microorganisms using different types of enzymes to catalyze substrates. In the case of enzymes that require a water-soluble environment to perform the reaction, however, the solubility of the substrate has a direct impact on the yield of the target chemical.

Since melanin in living organisms has excellent UV resistance and can be used as a black dye, it is one of the main target chemicals for current biosynthesis. In the biosynthesis of melanin, tyrosinase is the enzyme that produces melanin, and tyrosine is the substrate of tyrosinase. However, tyrosine has a water solubility of only 0.5 g/L at pH 7 which is the most suitable environment for the growth of microorganisms, resulting in an inability to further increase the efficiency of melanin biosynthesis. Accordingly, improving the utilization efficiency of tyrosine using microorganisms, and thereby improving the biosynthetic efficiency of melanin, is also a focus of the current research.

### SUMMARY

The present disclosure provides a genetically modified microorganism for enhancing melanin production efficiency, comprising: a first exogenous nucleic acid comprising a nucleic acid encoding a secreted L-amino acid ligase, wherein secreted L-amino acid ligase is capable of linking two amino acids to form a dipeptide; and a second exogenous nucleic acid comprising a nucleic acid encoding tyrosinase.

The present disclosure also provides a method for producing melanin. The method comprises culturing the foregoing genetically modified microorganism for enhancing melanin production efficiency in the presence of a first amino acid and a second amino acid, in the presence of a dipeptide, or in the presence of a first amino acid, a second amino acid and a dipeptide. The second amino acid is tyrosine. The dipeptide comprises a third amino acid and a fourth amino acid. The C-terminal of the third amino acid is linked to the N-terminal of the fourth amino acid. The third amino acid is the same as the first amino acid, while the fourth amino acid is the same as the second amino acid.

Moreover, the present disclosure also provides another method for producing melanin. The other method comprises culturing a genetically modified microorganism in the presence of a dipeptide. The dipeptide comprises a first amino acid and a second amino acid. The C-terminal of the first amino acid is linked to the N-terminal of the second amino acid. The second amino acid is tyrosine. The genetically modified microorganism comprises an exogenous nucleic acid, comprising a nucleic acid encoding tyrosinase.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1A shows the map of plasmid V-AT1;

FIG. 1B shows the map of plasmid V-AT2;

FIG. 1C shows the map of plasmid V-AT3;

FIG. 2A shows the results of high performance liquid chromatography (HPLC) for L-alanine standard (1.0 g/L), L-tyrosine standard (0.5 g/L) and L-alanyl-L-tyrosine standard (0.5 g/L);

FIG. 2B shows a standard curve of the concentration of L-alanine versus the peak area of L-alanine in high-performance liquid chromatography;

FIG. 2C shows a standard curve of the concentration of L-tyrosine versus the peak area of L-tyrosine in high-performance liquid chromatography;

FIG. 2D shows a standard curve of the concentration of dipeptide (L-alanyl-L-tyrosine) versus the peak area of dipeptide in high-performance liquid chromatography;

FIG. 3 shows a standard curve of melanin concentration versus OD 400;

FIG. 4 shows results of sodium dodecyl sulfate polyacrylamide gel electrophoresis for the supernatants (the cultured media) of the bacterial suspensions of the modified strains of Escherichia coli, JMD01 and JMD02;

FIG. 5 shows the concentrations of tyrosine in the supernatants (the cultured medium) of the bacterial suspensions of the modified strains of Escherichia coli, JMD01 and JMD02;

FIG. 6A shows the results of high-performance liquid chromatography for the supernatants (the cultured media) of the bacterial suspensions of the modified strains of Escherichia coli, JMD01 and JMD02. The dotted line is the signal curve of the standard while the solid line is the signal curve of the cultured medium (the sample) of the modified strain of Escherichia coli, JMD02. (Note: The supernatant (cultured medium) of the bacterial suspension of the modified strain of Escherichia coli, JMD01, does not show signal for dipeptide in high performance liquid chromatography.);

FIG. 6B shows the concentrations of dipeptide in the supernatants (the cultured media) of the bacterial suspensions of the modified strains of Escherichia coli, JMD01 and JMD02;

FIG. 7 shows the concentration of dipeptide in the supernatant (cultured medium) of the bacterial suspension of the modified strain of Escherichia coli, JMD02, under the addition of different concentrations of alanine;

FIG. 8 shows the melanin production of the modified strain of Escherichia coli, JMD03, cultured with the supernatant of the bacterial suspension of the modified strain of Escherichia coli, JMD02;

FIG. 9 shows the melanin production of the modified strains of Escherichia coli, JMD03 and JMD04; and

FIG. 10 shows the melanin production of the modified strain of Escherichia coli, JMD04, under the addition of different concentrations of tyrosine.

### DETAILED DESCRIPTION

The following description is made for the purpose of illustrating the general principles of the invention and should not be taken in a limiting sense. The scope of the invention is best determined by reference to the appended claims.

It is known that the solubility of the dipeptide of L-alanyl-L-tyrosine in water is more than 20 times that of tyrosine, and thus it is more easily utilized and ingested by microorganisms to increase the uptake of tyrosine thereby. It is confirmed in the present disclosure that the melanin production efficiency of microorganisms can be effectively improved through the use of dipeptides with better water solubility. More specifically, in the present disclosure, it is confirmed that compared to the extracellular amino acids (especially tyrosine), microorganisms can more effectively obtain the substrate (tyrosine) required for the production of melanin through the extracellular dipeptides with higher solubility in water (especially the dipeptides containing tyrosine), thereby improving the melanin production efficiency of the microorganisms.

According to the foregoing, the present disclosure may provide a genetically modified microorganism for enhancing melanin production efficiency, but it is not limited thereto. The genetically modified microorganism for enhancing melanin production efficiency of the present disclosure can achieve the effect of enhancing melanin production efficiency by itself through secreting an exogenous L-amino acid ligase to the outside of cell and extracellularly forming dipeptides using the amino acids present outside the cell, and ingesting and utilizing the formed dipeptides, without the need to add additional dipeptides during culture.

Meanwhile, a source microorganism of the genetically modified microorganism for enhancing melanin production efficiency of the present disclosure mentioned above may comprise, but is not limited to, a bacterium, an actinomycete, a yeast, a mold, etc.

In one embodiment, examples of the aforementioned bacterium that can be used as the source microorganism of the above-mentioned genetically modified microorganism for enhancing melanin production efficiency of the present disclosure may comprise a bacterium belonging to the genus Escherichia, a bacterium belonging to the genus Corynebacterium and a bacterium belonging to the genus Bacillus, but they are not limited thereto. Examples of a bacterium belonging to the genus Escherichia may comprise Escherichia coli, but they are not limited thereto. Examples of a bacterium belonging to the genus Corynebacterium may comprise, but are not limited to, Corynebacterium glutamicum. Examples of a bacterium belonging to the genus Bacillus may comprise Bacillus subtilis, but they are not limited thereto.

In another embodiment, the above-mentioned bacterium that can be used as the source microorganism of the above-mentioned genetically modified microorganism for enhancing melanin production efficiency of the present disclosure may comprise, but is not limited to, Escherichia coli, Corynebacterium glutamicum, Bacillus subtilis, etc. In one specific embodiment, the above-mentioned bacterium that can be used as the source microorganism of the above-mentioned genetically modified microorganism for enhancing melanin production efficiency of the present disclosure may be Escherichia coli. Furthermore, examples of above-mentioned Escherichia coli may comprise Escherichia coli BL21, BW25113, K12, DH5α, XL1-blue, but they are not limited thereto.

In one embodiment, examples of the aforementioned actinomycete that can be used as the source microorganism of the above-mentioned genetically modified microorganism for enhancing melanin production efficiency of the present disclosure may comprise, but are not limited to, an actinomycete belonging to the genus Streptomyces. Examples of an actinomycete belonging to the genus Streptomyces may comprise Streptomyces lividans, Streptomyces violaceoruber and Streptomyces coelicolor, but they are not limited thereto.

In another embodiment, the above-mentioned actinomycete that can be used as the source microorganism of the above-mentioned genetically modified microorganism for enhancing melanin production efficiency of the present disclosure may comprise, but is not limited to, Streptomyces lividans, Streptomyces violaceoruber, Streptomyces coelicolor, etc.

In one embodiment, examples of the aforementioned yeast that can be used as the source microorganism of the above-mentioned genetically modified microorganism for enhancing melanin production efficiency of the present disclosure may comprise, but are not limited to, a yeast belonging to the genus Yarrowia, a yeast belonging to the genus Saccharomyces and a yeast belonging to the genus Pichia. Examples of a yeast belonging to the genus Yarrowia may comprise Yarrowia lipolytica, but they are not limited thereto. Examples of a yeast belonging to the genus Saccharomyces may comprise, but are not limited to, Saccharomyces cerevisiae. Examples of a yeast belonging to the genus Pichia may comprise Pichia pastoris, but they are not limited thereto.

In another embodiment, the above-mentioned yeast that can be used as the source microorganism of the above-mentioned genetically modified microorganism for enhancing melanin production efficiency of the present disclosure may comprise, but is not limited to, Yarrowia lipolytica, Saccharomyces cerevisiae, Pichia pastoris, etc.

In one embodiment, examples of the aforementioned mold that can be used as the source microorganism of the above-mentioned genetically modified microorganism for enhancing melanin production efficiency of the present disclosure may comprise a mold belonging to the genus Aspergillus, but they are not limited thereto. Examples of an actinomycete belonging to the genus Aspergillus may comprise Aspergillus oryzae and Aspergillus terreus, but they are not limited thereto.

In another embodiment, the above-mentioned mold that can be used as the source microorganism of the above-mentioned genetically modified microorganism for enhancing melanin production efficiency of the present disclosure may comprise, but is not limited to, Aspergillus oryzae, Aspergillus terreus, etc.

Moreover, the genetically modified microorganism for enhancing melanin production efficiency of the present disclosure may comprise, but is not limited to, a first exogenous nucleic acid and a second exogenous nucleic acid. The first exogenous nucleic acid mentioned above may comprise a nucleic acid encoding a secreted L-amino acid ligase, but it is not limited thereto. The second exogenous nucleic acid mentioned above may comprise, but is not limited to, a nucleic acid encoding tyrosinase.

The secreted L-amino acid ligase mentioned above may be secreted extracellularly by microorganisms and may be capable of linking two amino acids to form a dipeptide, but is not limited thereto. Furthermore, the secreted L-amino acid ligase mentioned above may link arbitrary two amino acids, without particular limitations. In one embodiment, the secreted L-amino acid ligase mentioned above may link the C-terminal of a first amino acid to the N-terminal of a second amino acid while both of the first amino acid and the second amino acid may be arbitrary amino acids, without particular limitations. In one specific embodiment, the secreted L-amino acid ligase mentioned above may link the C-terminal of a first amino acid to the N-terminal of a second amino acid, and the second amino acid may be tyrosine. In addition, in one further specific embodiment, the secreted L-amino acid ligase mentioned above may link the C-terminal of a first amino acid to the N-terminal of a second amino acid, and the second amino acid may be tyrosine while the first amino acid may be alanine.

In other words, the dipeptide formed by the secreted L-amino acid ligase mentioned above may comprise arbitrary two amino acids linked to each other, and is not particularly limited. In one embodiment, the dipeptide formed by the secreted L-amino acid ligase mentioned above may comprise a first amino acid and a second, and the C-terminal of a first amino acid is linked the N-terminal of a second amino acid while both of the first amino acid and the second amino acid may be arbitrary amino acids, without particular limitations. In one specific embodiment, the dipeptide formed by the secreted L-amino acid ligase mentioned above may comprise a first amino acid and a second, and the C-terminal of a first amino acid is linked the N-terminal of a second amino acid while the second amino acid may be tyrosine, without particular limitations. Moreover, in one further specific embodiment, the dipeptide formed by the secreted L-amino acid ligase mentioned above may comprise a first amino acid and a second, and the C-terminal of a first amino acid is linked the N-terminal of a second amino acid while the second amino acid may be tyrosine and the first amino acid may be alanine, namely, the dipeptide is L-alanyl-L-tyrosine.

The sequence of the foregoing nucleic acid encoding a secreted L-amino acid ligase in the foregoing first exogenous nucleic acid may comprise, but is not limited to, a sequence having 85% or more sequence identity with the sequence of SEQ ID NO. 1. In one specific embodiment, the sequence of the foregoing nucleic acid encoding a secreted L-amino acid ligase may comprise the sequence of SEQ ID NO. 1.

The term “having 85% or more sequence identity with” used in the present disclosure means that there is an identity of about 85%-100% between two sequences, such as about 85%, about 90%, about 95%, about 96%, about 97%, about 98%, about 99%, about 99.5%, about 99.8%, about 100%, but it is not limited thereto.

In one embodiment, the sequence of the foregoing nucleic acid encoding a secreted L-amino acid ligase may be derived from Serratia symbiotica, but it is not limited thereto.

In the foregoing embodiment in which the sequence of the foregoing nucleic acid encoding a secreted L-amino acid ligase may be derived from Serratia symbiotica, in one specific embodiment, the sequence of the foregoing nucleic acid encoding a secreted L-amino acid ligase may comprise the sequence of SEQ ID NO. 1.

Moreover, the sequence of the foregoing nucleic acid encoding tyrosinase in the foregoing second exogenous nucleic acid may comprise, a sequence having 85% or more sequence identity with the sequence of SEQ ID NO. 2, but it is not limited thereto. In one specific embodiment, the sequence of the foregoing nucleic acid encoding tyrosinase may comprise the sequence of SEQ ID NO. 2.

In one embodiment, the sequence of the foregoing nucleic acid encoding tyrosinase may be derived from Ralstonia pseudosolanacearum, but it is not limited thereto.

In the foregoing embodiment in which the sequence of the foregoing nucleic acid encoding tyrosinase may be derived from Ralstonia pseudosolanacearum, in one specific embodiment, the sequence of the foregoing nucleic acid encoding tyrosinase may comprise the sequence of SEQ ID NO. 2.

The location of the foregoing first exogenous nucleic acid contained in the genetically modified microorganism for enhancing melanin production efficiency of the present disclosure has no particular limitation, as long as the foregoing first exogenous nucleic acid can display its intended function, such as encoding a protein that conforms to the sequence information thereof. For example, the foregoing first exogenous nucleic acid contained in the genetically modified microorganism for enhancing melanin production efficiency of the present disclosure may be located in an expression vector or may be located in the genome of the genetically modified microorganism for enhancing melanin production efficiency of the present disclosure, but it is not limited thereto.

Similarly, the location of the foregoing second exogenous nucleic acid contained in the genetically modified microorganism for enhancing melanin production efficiency of the present disclosure also has no particular limitation, as long as the foregoing second exogenous nucleic acid can display its intended function, such as encoding a protein that conforms to the sequence information thereof. For example, the foregoing second exogenous nucleic acid contained in the genetically modified microorganism for enhancing melanin production efficiency of the present disclosure also may be located in an expression vector or may be located in the genome of the genetically modified microorganism for enhancing melanin production efficiency of the present disclosure, but it is not limited thereto.

In one embodiment, the foregoing first exogenous nucleic acid contained in the genetically modified microorganism for enhancing melanin production efficiency of the present disclosure may be located in a first expression vector while the foregoing second exogenous nucleic acid contained in the genetically modified microorganism for enhancing melanin production efficiency of the present disclosure may be located in a second expression vector. The first expression vector and the second expression vector mentioned above may independently comprise, a plasmid, a cosmid, a viral vector, a chromosome (artificial chromosome), etc., but they are not limited thereto.

In one specific embodiment, the foregoing first exogenous nucleic acid contained in the genetically modified microorganism for enhancing melanin production efficiency of the present disclosure may exist in a first expression vector, and the first expression vector mentioned above is a plasmid, meanwhile, the foregoing second exogenous nucleic acid contained in the genetically modified microorganism for enhancing melanin production efficiency of the present disclosure may exist in a second expression vector, and the second expression vector mentioned above is another plasmid.

In addition, the genetically modified microorganism for enhancing melanin production efficiency of the present disclosure mentioned above may be a genetically modified strain of Escherichia coli, BL-21 JMD04, which deposited in the German Collection of Microorganisms and Cell Cultures GmbH (DSMZ) on Nov. 4, 2024, with the deposit number of DSM 35223, and which is also deposited in Bioresource Collection and Research Centre (BCRC) of Food Industry Research and Development Institute (FIRDI) (ROC.) on Nov. 5, 2024, with the deposit number of BCRC 940702.

Furthermore, the present disclosure also provides a method for producing melanin.

The method for producing melanin may comprise a following step, but it is not limited thereto.

First, a genetically modified microorganism for producing melanin is cultured in the presence of a first amino acid and a second amino acid, in the presence of a dipeptide, or in the presence of a first amino acid, a second amino acid and a dipeptide. The genetically modified microorganism for producing melanin mentioned above may be any genetically modified microorganism for enhancing melanin production efficiency of the present disclosure mentioned above. Moreover, the second amino acid mentioned above may be tyrosine. The dipeptide mentioned above may comprise a third amino acid and a fourth amino acid, and the C-terminal of the third amino acid is linked to the N-terminal of the fourth amino acid, and the third amino acid is the same as the first amino acid while the fourth amino acid is the same as the second amino acid.

For the method for producing melanin of the present disclosure, under the condition that it is in the presence of a first amino acid and a second amino acid, in one embodiment, the concentration of the first amino acid mentioned above may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto. Moreover, in one specific embodiment, the first amino acid mentioned above may be alanine, and the concentration of the first amino acid mentioned above may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto.

For the method for producing melanin of the present disclosure, under the condition that it is in the presence of a first amino acid and a second amino acid, in one embodiment, the concentration of the second amino acid mentioned above may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto. Furthermore, in one specific embodiment, the first amino acid mentioned above may be alanine, and the concentration of the second amino acid mentioned above may be, but is not limited to, about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L.

In addition, for the method for producing melanin of the present disclosure, under the condition that it is in the presence of a dipeptide, in one embodiment, the concentration of the dipeptide may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto. In one specific embodiment, the third amino acid in the dipeptide mentioned above may be alanine, and the concentration of the dipeptide mentioned above may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto.

For the method for producing melanin of the present disclosure, under the condition that it is in the presence of a first amino acid, a second amino acid and a dipeptide, in one embodiment, the concentrations of the first amino acid, the second amino acid and the dipeptide mentioned above may independently be about 0.05-25 g/L, such as about 0.1-22.5 g/L, about 0.15-20 g/L, about 0.2-17.5 g/L, about 0.25-15 g/L, about 0.5-12.5 g/L, about 1.5-10 g/L, about 2-7.5 g/L, about 2.5-5 g/L, about 0.05 g/L, about 0.1 g/L, about 0.125 g/L, about 0.15 g/L, about 0.2 g/L, about 0.25 g/L, about 0.3 g/L, about 0.35 g/L, about 0.4 g/L, about 0.45 g/L, about 0.5 g/L, about 1 g/L, about 1.25 g/L, about 1.5 g/L, about 2 g/L, about 2.5 g/L, about 3 g/L, about 3.5 g/L, about 4 g/L, about 4.5 g/L, about 5 g/L, about 7.5 g/L, about 10 g/L, about 12.5 g/L, about 15 g/L, about 17.5 g/L, about 20 g/L, about 22.5 g/L, about 25 g/L, but it is not limited thereto. In one specific embodiment, the first amino acid mentioned above may be alanine, and the concentrations of the first amino acid, the second amino acid and the dipeptide mentioned above may independently be about 0.05-25 g/L, such as about 0.1-22.5 g/L, about 0.15-20 g/L, about 0.2-17.5 g/L, about 0.25-15 g/L, about 0.5-12.5 g/L, about 1.5-10 g/L, about 2-7.5 g/L, about 2.5-5 g/L, about 0.05 g/L, about 0.1 g/L, about 0.125 g/L, about 0.15 g/L, about 0.2 g/L, about 0.25 g/L, about 0.3 g/L, about 0.35 g/L, about 0.4 g/L, about 0.45 g/L, about 0.5 g/L, about 1 g/L, about 1.25 g/L, about 1.5 g/L, about 2 g/L, about 2.5 g/L, about 3 g/L, about 3.5 g/L, about 4 g/L, about 4.5 g/L, about 5 g/L, about 7.5 g/L, about 10 g/L, about 12.5 g/L, about 15 g/L, about 17.5 g/L, about 20 g/L, about 22.5 g/L, about 25 g/L, but it is not limited thereto.

In the method for producing melanin of the present disclosure, in one embodiment, the above-mentioned genetically modified microorganism for producing melanin may be cultured at about 25-40° C., such as about 25° C., about 26° C., about 27° C., about 28° C., about 29° C., about 30° C., about 32° C., about 34° C., about 35° C., about 36° C., about 37° C., about 37.5° C., about 38° C., about 39° C., about 40° C., but it is not limited thereto.

Moreover, in the method for producing melanin of the present disclosure, in one embodiment, the above-mentioned genetically modified microorganism for producing melanin may be cultured at about pH 6.0-8.0, such as about pH 6.0, about pH 6.2, about pH 6.3, about pH 6.5, about pH 6.8, about pH 7.0, about pH 7.2, about pH 7.4, about pH 7.5, about pH 7.8, about pH 8.0, but it is not limited thereto.

Furthermore, in the method for producing melanin of the present disclosure, in one embodiment, the above-mentioned genetically modified microorganism for producing melanin may be cultured for about 1-90 hours, such as about 2-84 hours, about 4-78 hours, about 6-72 hours, about 12-66 hours, about 1 hours, about 2 hours, about 3 hours, about 4 hours, about 5 hours, about 6 hours, about 7 hours, about 8 hours, about 9 hours, about 10 hours, about 11 hours, about 12 hours, about 15 hours, about 16 hours, about 18 hours, about hours, about 24 hours, about 27 hours, about 30 hours, about 33 hours, about 36 hours, about 39 hours, about 40 hours, about 42 hours, about 45 hours, about 48 hours, about 50 hours, about 54 hours, about 60 hours, about 66 hours, about 72 hours, about 78 hours, about hours, about 90 hours, but it is not limited thereto.

In addition, in one embodiment, in the method for producing melanin of the present disclosure mentioned above, in the presence of a first amino acid and a second amino acid, in the presence of a dipeptide, or in the presence of a first amino acid, a second amino acid and a dipeptide, the step of culturing the genetically modified microorganism for producing melanin may comprise a first culture procedure and a second culture procedure, but it is not limited thereto. In this embodiment, the temperature for the second culture procedure mentioned above is lower than that for the first culture procedure mentioned above and is performed after the first culture procedure mentioned above is completed, and the time for performing the first culture procedure mentioned above plus the time for performing the second culture procedure mentioned above is the time for culturing the genetically modified microorganism for producing melanin in the method for producing melanin of the present disclosure.

Furthermore, in the foregoing embodiment in which in the method for producing melanin of the present disclosure, in the presence of a first amino acid and a second amino acid, in the presence of a dipeptide, or in the presence of a first amino acid, a second amino acid and a dipeptide, the step of culturing the genetically modified microorganism for producing melanin may comprise a first culture procedure and a second culture procedure, the temperature for the first culture procedure may be about 25.5-40° C., such as about 25.5° C., about 26° C., about 27° C., about 28° C., about 29° C., about 30° C., about 32° C., about 34° C., about 35° C., about 36° C., about 37° C., about 37.5° C., about 38° C., about 39° C., about 40° C., but it is bot limited thereto while the temperature for the second culture procedure may be about 25-39.5° C., such as about 25° C., about 26° C., about 27° C., about 28° C., about 29° C., about 30° C., about 32° C., about 34° C., about 35° C., about 36° C., about 37° C., about 37.5° C., about 38° C., about 39° C., about 39.5° C., but it is not limited thereto. In one specific embodiment, the temperature for the first culture procedure may be about 37° C. while the temperature for the second culture procedure may be about 30° C.

Moreover, in the foregoing embodiment in which in the method for producing melanin of the present disclosure, in the presence of a first amino acid and a second amino acid, in the presence of a dipeptide, or in the presence of a first amino acid, a second amino acid and a dipeptide, the step of culturing the genetically modified microorganism for producing melanin may comprise a first culture procedure and a second culture procedure, the pH values for the first culture procedure and the second procedure may independently be about pH 6.0-8.0, such as about pH 6.0, about pH 6.2, about pH 6.3, about pH 6.5, about pH 6.8, about pH 7.0, about pH 7.2, about pH 7.4, about pH 7.5, about pH 7.8, about pH 8.0, but it is not limited thereto. In one specific embodiment, the pH values for the first culture procedure and the second culture procedure may both be about pH 7.0.

In the foregoing embodiment in which in the method for producing melanin of the present disclosure, in the presence of a first amino acid and a second amino acid, in the presence of a dipeptide, or in the presence of a first amino acid, a second amino acid and a dipeptide, the step of culturing the genetically modified microorganism for producing melanin may comprise a first culture procedure and a second culture procedure, the time for performing the first culture procedure may be about 2-12 hours, such as 4-8 hours, about 2 hours, about 3 hours, about 4 hours, about 5 hours, about 6 hours, about 7 hours, about 8 hours, about 9 hours, about 10 hours, about 11 hours, about 12 hours, but it is not limited thereto while the time for performing the second culture procedure may be about 8-72 hours, such as about 10-70 hours, about 12-66 hours, about 18-60 hours, about 24-54 hours, about hours, about 9 hours, about 10 hours, about 12 hours, about 18 hours, about 24 hours, about hours, about 36 hours, about 42 hours, about 48 hours, about 54 hours, about 60 hours, about 66 hours, about 72 hours, but it is not limited thereto. In one specific embodiment, the time for performing the first culture procedure may be about 6 hours while the second culture procedure may be about 40 hours.

In addition, in the method for producing melanin of the present disclosure, a medium used to culture the genetically modified microorganism for producing melanin mentioned above has no particular limitation, as long as the genetically modified microorganism for producing melanin mentioned above can grow therein. In one embodiment, in the method for producing melanin of the present disclosure, the medium used to culture the genetically modified microorganism for producing melanin mentioned above may be lysogeny broth (Luria Bertani broth, LB) medium, a phosphate buffer (PB) with glucose and yeast extract (YE), etc., but it is not limited thereto. In one specific embodiment, the medium used to culture the genetically modified microorganism for producing melanin mentioned above may be lysogeny broth medium. In another specific embodiment, the medium used to culture the genetically modified microorganism for producing melanin mentioned above may be a phosphate buffer with about 5-15 g/L glucose and about 1-10 g/L yeast extract, such as a phosphate buffer with about 10 g/L glucose and about 5 g/L yeast extract.

Furthermore, in the method for producing melanin of the present disclosure, a medium used to culture the genetically modified microorganism for producing melanin mentioned above may originally contain any one, two or all of the above-mentioned first amino acid, second amino acid and dipeptide, or may be additionally supplemented with any one, two or all of the above-mentioned first amino acid, second amino acid and dipeptide.

Moreover, in another embodiment, the method for producing melanin of the present disclosure may further comprise extracting melanin from the genetically modified microorganism for producing melanin mentioned above after completing the foregoing step of culturing a genetically modified microorganism for producing melanin in the presence of a first amino acid and a second amino acid, in the presence of a dipeptide, or in the presence of a first amino acid, a second amino acid and a dipeptide, it is not limited thereto.

The manner for extracting melanin from the genetically modified microorganism for producing melanin mentioned above has no particular limitation, as long as the melanin produced by the microorganism can be separated from the microorganism. For example, the microorganism can be cell disrupted first and then centrifuged to obtain the melanin exist in the supernatant, but it is not limited thereto.

Moreover, in one specific embodiment, the genetically modified microorganism for producing melanin used in the method for producing melanin of the present disclosure may be a genetically modified strain of Escherichia coli, BL-21 JMD04, which deposited in the German Collection of Microorganisms and Cell Cultures GmbH (DSMZ) on Nov. 4, 2024, with the deposit number of DSM 35223, and which is also deposited in Bioresource Collection and Research Centre (BCRC) of Food Industry Research and Development Institute (FIRDI) (ROC.) on Nov. 5, 2024, with the deposit number of BCRC 940702.

In addition, based on the foregoing, the present disclosure may further provide another method for producing melanin. The other method for producing melanin of the present disclosure mentioned above may comprise, but is not limited to, a following step.

First, a genetically modified microorganism is cultured in the presence of a dipeptide.

The dipeptide mentioned above may comprise a first amino acid and a second amino acid, and the C-terminal of the first amino acid is linked to the N-terminal of the second amino acid while the second amino acid may be tyrosine. In one embodiment, the dipeptide mentioned above may comprise a first amino acid and a second amino acid, and the C-terminal of a first amino acid is linked the N-terminal of a second amino acid while the second amino acid may be tyrosine and the first amino acid may be alanine, namely, the dipeptide is L-alanyl-L-tyrosine.

Moreover, in the other method for producing melanin of the present disclosure mentioned above, the concentration of the dipeptide mentioned above may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto. In one specific embodiment, the first amino acid in the dipeptide mentioned above may be alanine, and the concentration of the dipeptide may be about 0.1-50 g/L, such as about 0.2-45 g/L, about 0.3-40 g/L, about 0.4-35 g/L, about 0.5-30 g/L, about 1-25 g/L, about 3-20 g/L, about 4-15 g/L, about 5-10 g/L, about 0.1 g/L, about 0.2 g/L, about 0.25 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 15 g/L, about 20 g/L, about 25 g/L, about 30 g/L, about 35 g/L, about 40 g/L, about 45 g/L, about 50 g/L, but it is not limited thereto.

Meanwhile, in the above-mentioned other method for producing melanin of the present disclosure, a source microorganism of the above-mentioned genetically modified microorganism may comprise, but is not limited to, a bacterium, an actinomycete, a yeast, a mold, etc.

In one embodiment, examples of the aforementioned bacterium that can be used as the source microorganism of the above-mentioned genetically modified microorganism in the other method for producing melanin of the present disclosure, may comprise a bacterium belonging to the genus Escherichia, a bacterium belonging to the genus Corynebacterium and a bacterium belonging to the genus Bacillus, but they are not limited thereto. Examples of a bacterium belonging to the genus Escherichia may comprise Escherichia coli, but they are not limited thereto. Examples of a bacterium belonging to the genus Corynebacterium may comprise, but are not limited to, Corynebacterium glutamicum. Examples of a bacterium belonging to the genus Bacillus may comprise Bacillus subtilis, but they are not limited thereto.

In another embodiment, the above-mentioned bacterium that can be used as the source microorganism of the above-mentioned genetically modified microorganism in the other method for producing melanin of the present disclosure may comprise, but is not limited to, Escherichia coli, Corynebacterium glutamicum, Bacillus subtilis, etc. In one specific embodiment, the above-mentioned bacterium that can be used as the source microorganism of the above-mentioned genetically modified microorganism in the other method for producing melanin of the present disclosure may be Escherichia coli. Furthermore, examples of above-mentioned Escherichia coli may comprise Escherichia coli BL21, BW25113, K12, DH5α, XL1-blue, but they are not limited thereto.

In one embodiment, examples of the aforementioned actinomycete that can be used as the source microorganism of the above-mentioned genetically modified microorganism in the other method for producing melanin of the present disclosure may comprise, but are not limited to, an actinomycete belonging to the genus Streptomyces. Examples of an actinomycete belonging to the genus Streptomyces may comprise Streptomyces lividans, Streptomyces violaceoruber and Streptomyces coelicolor, but they are not limited thereto.

In another embodiment, the above-mentioned actinomycete that can be used as the source microorganism of the above-mentioned genetically modified microorganism in the other method for producing melanin of the present disclosure may comprise, but is not limited to, Streptomyces lividans, Streptomyces violaceoruber, Streptomyces coelicolor, etc.

In one embodiment, examples of the aforementioned yeast that can be used as the source microorganism of the above-mentioned genetically modified microorganism in the other method for producing melanin of the present disclosure, may comprise, but are not limited to, a yeast belonging to the genus Yarrowia, a yeast belonging to the genus Saccharomyces and a yeast belonging to the genus Pichia. Examples of a yeast belonging to the genus Yarrowia may comprise Yarrowia lipolytica, but they are not limited thereto. Examples of a yeast belonging to the genus Saccharomyces may comprise, but are not limited to, Saccharomyces cerevisiae. Examples of a yeast belonging to the genus Pichia may comprise Pichia pastoris, but they are not limited thereto.

In another embodiment, the above-mentioned yeast that can be used as the source microorganism of the above-mentioned genetically modified microorganism in the other method for producing melanin of the present disclosure may comprise, but is not limited to, Yarrowia lipolytica, Saccharomyces cerevisiae, Pichia pastoris, etc.

In one embodiment, examples of the aforementioned mold that can be used as the source microorganism of the above-mentioned genetically modified microorganism in the other method for producing melanin of the present disclosure may comprise a mold belonging to the genus Aspergillus, but they are not limited thereto. Examples of an actinomycete belonging to the genus Aspergillus may comprise Aspergillus oryzae and Aspergillus terreus, but they are not limited thereto.

In another embodiment, the above-mentioned mold that can be used as the source microorganism of the above-mentioned genetically modified microorganism in the other method for producing melanin of the present disclosure may comprise, but is not limited to, Aspergillus oryzae, Aspergillus terreus, etc.

Furthermore, in the above-mentioned other method for producing melanin of the present disclosure, the above-mentioned genetically modified microorganism may comprise, but is not limited to, an exogenous nucleic acid. The exogenous nucleic acid mentioned above may comprise a nucleic acid encoding tyrosinase, but it is not limited thereto.

The sequence of the foregoing nucleic acid encoding tyrosinase in the foregoing exogenous nucleic acid may comprise, a sequence having 85% or more sequence identity with the sequence of SEQ ID NO. 2, but it is not limited thereto. In one specific embodiment, the sequence of the foregoing nucleic acid encoding tyrosinase in the foregoing exogenous nucleic acid may comprise the sequence of SEQ ID NO. 2.

In one embodiment, the sequence of the foregoing nucleic acid encoding tyrosinase may be derived from Ralstonia pseudosolanacearum, but it is not limited thereto.

In the foregoing embodiment in which the sequence of the foregoing nucleic acid encoding tyrosinase may be derived from Ralstonia pseudosolanacearum, in one specific embodiment, the sequence of the foregoing nucleic acid encoding tyrosinase may comprise the sequence of SEQ ID NO. 2.

The location of the above-mentioned exogenous nucleic acid contained in the above-mentioned genetically modified microorganism in the other method for producing melanin of the present disclosure also has no particular limitation, as long as the above-mentioned exogenous nucleic acid can display its intended function, such as encoding a protein that conforms to the sequence information thereof. For example, the above-mentioned exogenous nucleic acid contained in the above-mentioned genetically modified microorganism in the other method for producing melanin of the present disclosure may be located in an expression vector or may be located in the genome of the above-mentioned genetically modified microorganism in the other method for producing melanin of the present disclosure, but it is not limited thereto.

In one embodiment, the above-mentioned exogenous nucleic acid contained in the above-mentioned genetically modified microorganism in the other method for producing melanin of the present disclosure may be located in an expression vector. The above-mentioned expression vector may comprise, a plasmid, a cosmid, a viral vector, a chromosome (artificial chromosome), etc., but it is not limited thereto. In one specific embodiment, the above-mentioned expression vector is a plasmid.

In the other method for producing melanin of the present disclosure mentioned above, in one embodiment, the genetically modified microorganism mentioned above may be cultured at about 25-40° C., such as about 25° C., about 26° C., about 27° C., about 28° C., about 29° C., about 30° C., about 32° C., about 34° C., about 35° C., about 36° C., about 37° C., about 37.5° C., about 38° C., about 39° C., about 40° C., but it is not limited thereto.

Moreover, in the other method for producing melanin of the present disclosure mentioned above, in one embodiment, the genetically modified microorganism mentioned above may be cultured at about pH 6.0-8.0, such as about pH 6.0, about pH 6.2, about pH 6.3, about pH 6.5, about pH 6.8, about pH 7.0, about pH 7.2, about pH 7.4, about pH 7.5, about pH 7.8, about pH8.0, but it is not limited thereto.

Furthermore, in the other method for producing melanin of the present disclosure mentioned above, in one embodiment, the genetically modified microorganism mentioned above may be cultured for about 1-90 hours, such as about 2-84 hours, about 4-78 hours, about 6-72 hours, about 12-66 hours, about 1 hours, about 2 hours, about 3 hours, about 4 hours, about 5 hours, about 6 hours, about 7 hours, about 8 hours, about 9 hours, about 10 hours, about 11 hours, about 12 hours, about 15 hours, about 16 hours, about 18 hours, about hours, about 24 hours, about 27 hours, about 30 hours, about 33 hours, about 36 hours, about 39 hours, about 40 hours, about 42 hours, about 45 hours, about 48 hours, about 50 hours, about 54 hours, about 60 hours, about 66 hours, about 72 hours, about 78 hours, about hours, about 90 hours, but it is not limited thereto.

In one embodiment, in the other method for producing melanin of the present disclosure mentioned above, in the presence of a dipeptide, the step of culturing the genetically modified microorganism may comprise a first culture procedure and a second culture procedure, but it is not limited thereto. In the embodiment which comprise the first culture procedure and the second culture procedure, the temperature for the second culture procedure mentioned above is lower than that for the first culture procedure mentioned above, and the second culture procedure is performed after the first culture procedure mentioned above is completed. As for the culture conditions for the first culture procedure and the culture conditions for the second culture procedure, please refer to the description for the step of culturing genetically modified microorganisms described in the other method of producing melanin of the present disclosure as shown in below.

Furthermore, in the foregoing embodiment in which in the other method for producing melanin of the present disclosure, in the presence of a dipeptide, the step of culturing the genetically modified microorganism may comprise a first culture procedure and a second culture procedure, the temperature for the first culture procedure may be about 25.5-40° C., such as 25.5° C., about 26° C., about 27° C., about 28° C., about 29° C., about 30° C., about 32° C., about 34° C., about 35° C., about 36° C., about 37° C., about 37.5° C., about 38° C., about 39° C., about 40° C., but it is bot limited thereto while the temperature for the second culture procedure may be about 25-39.5° C., such as 25° C., about 26° C., about 27° C., about 28° C., about 29° C., about 30° C., about 32° C., about 34° C., about 35° C., about 36° C., about 37° C., about 37.5° C., about 38° C., about 39° C., about 39.5° C., but it is not limited thereto. In one specific embodiment, the temperature for the first culture procedure may be about 37° C. while the temperature for the second culture procedure may be about 30° C.

In addition, in the foregoing embodiment in which in the other method for producing melanin of the present disclosure, in the presence of a dipeptide, the step of culturing the genetically modified microorganism may comprise a first culture procedure and a second culture procedure, the pH values for the first culture procedure and the second procedure may independently be about pH 6.0-8.0, such as about pH 6.0, about pH 6.2, about pH 6.3, about pH 6.5, about pH 6.8, about pH 7.0, about pH 7.2, about pH 7.4, about pH 7.5, about pH 7.8, about pH 8.0, but it is not limited thereto. In one specific embodiment, the pH values for the first culture procedure and the second procedure may both be about pH 7.0.

In the foregoing embodiment in which in the other method for producing melanin of the present disclosure, in the presence of a dipeptide, the step of culturing the genetically modified microorganism may comprise a first culture procedure and a second culture procedure, the time for performing the first culture procedure may be about 2-12 hours, such as about 4-8 hours, about 2 hours, about 3 hours, about 4 hours, about 5 hours, about 6 hours, about 7 hours, about 8 hours, about 9 hours, about 10 hours, about 11 hours, about 12 hours, but it is not limited thereto while the time for performing the second culture procedure may be about 8-72 hours, such as about 10-70 hours, about 12-66 hours, about 18-60 hours, about 24-54 hours, about 8 hours, about 9 hours, about 10 hours, about 12 hours, about 18 hours, about 24 hours, about 30 hours, about 36 hours, about 42 hours, about 48 hours, about 54 hours, about 60 hours, about 66 hours, about 72 hours, but it is not limited thereto. In one specific embodiment, the time for performing the first culture procedure may be about 6 hours while the second culture procedure may be about 40 hours.

In addition, in the other method for producing melanin of the present disclosure mentioned above, a medium used to culture the genetically modified microorganism mentioned above has no particular limitation, as long as the genetically modified microorganism mentioned above can grow therein. In one embodiment, in the other method for producing melanin of the present disclosure mentioned above, the medium used to culture the genetically modified microorganism mentioned above may be lysogeny broth medium, a phosphate buffer with glucose and yeast extract, etc., but it is not limited thereto. In one specific embodiment, the medium used to culture the genetically modified microorganism mentioned above may be lysogeny broth medium. In another specific embodiment, the medium used to culture the genetically modified microorganism mentioned above may be a phosphate buffer with about 5-15 g/L glucose and about 1-10 g/L yeast extract, such as a phosphate buffer with about 10 g/L glucose and about 5 g/L yeast extract.

Furthermore, in the other method for producing melanin of the present disclosure mentioned above, a medium used to culture the genetically modified microorganism mentioned above may originally contain the dipeptide mentioned above, or may be additionally supplemented with the dipeptide mentioned above.

Moreover, in another embodiment, the other method for producing melanin of the present disclosure mentioned above may further comprise extracting melanin from the genetically modified microorganism mentioned above after completing the foregoing step of culturing a genetically modified microorganism in the presence of a dipeptide, but it is not limited thereto.

The manner for extracting melanin from the genetically modified microorganism mentioned above has no particular limitation, as long as the melanin produced by the microorganism can be separated from the microorganism. For example, the microorganism can be cell disrupted first and then centrifuged to obtain the melanin exist in the supernatant, but it is not limited thereto.

### EXAMPLES

### A. Material and Methods

1. Genetic Modification for Escherichia coli

### (1) Construction of Plasmids V-AT1, V-AT2 and V-AT3

### (i) Construction of Plasmid V-AT1

The nucleic acid sequence encoding a secreted L-amino acid ligase derived from the genome of Serratia symbiotica, the sequence of SEQ ID NO. 1 (the amino acid sequence encoded thereby is the sequence of SEQ ID NO. 4), was obtained from the National Center for Biotechnology Information (NCBI) database. This secreted L-amino acid ligase can link the C-terminal of an amino acid to the N-terminal of another amino acid to form a dipeptide, especially can form a dipeptide of L-alanyl-L-tyrosine.

For the foregoing nucleic acid sequence encoding a secreted L-amino acid ligase (the sequence of SEQ ID NO. 1), primers for polymerase chain reaction (as shown in the following Table 1) and restriction enzyme cutting sites were designed to meet the requirements of multiple cloning sites of plasmid.

TABLE 1
Primers for polymerase chain reaction
designed for the foregoing nucleic acid
sequence encoding a secreted L-amino acid
ligase (the sequence of SEQ ID NO. 1)

| Primer | Sequence | SEQ ID NO. |
|---|---|---|
| Forward | TGCTGGTCTGCTGCTCCTCGCTGCCCAG | SEQ ID |
| primer | CCGGCGATGGCCATGTCTGTTATCGCGG | NO. 7 |
| | TTCT | |
| Reverse | CTTTGTTAGCAGCCGGATCTCAGTGGTG | |
| primer | GTGGTGGTGGTGCTCGAGAGACAGCGC | SEQ ID |
| | GCTCT | NO. 8 |

The foregoing nucleic acid sequence encoding a secreted L-amino acid ligase (the sequence of SEQ ID NO. 1) was amplified through a polymerase chain reaction using the above-mentioned primers, and then the amplified DNA fragment was purified by electrophoresis. The obtained purified DNA fragment was ligated to plasmid pET-26b via specific restriction enzymes to obtain plasmid V-AT1.

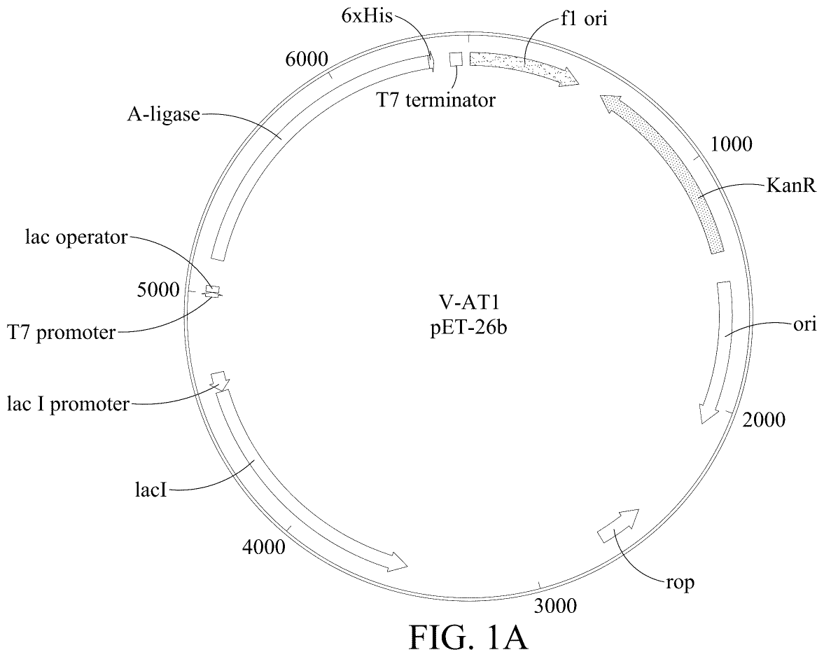

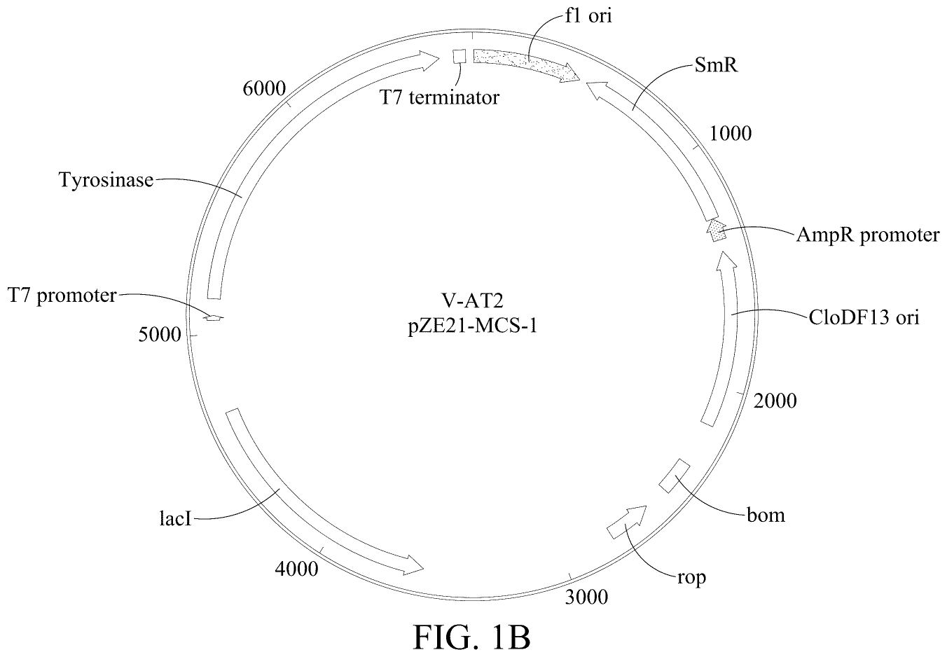

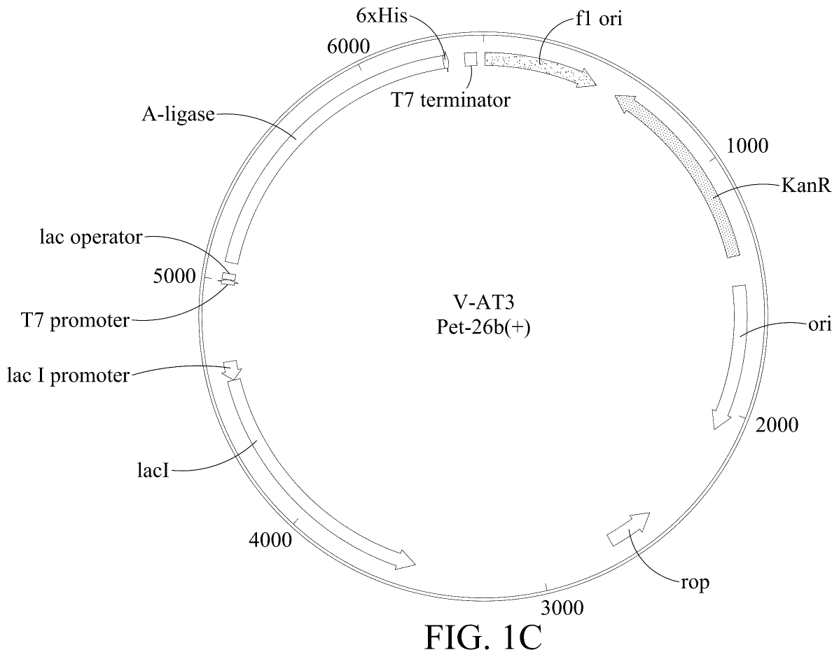

The map of plasmid V-AT1 is shown in FIG. 1A, which carries the nucleic acid sequence encoding a secreted L-amino acid ligase (the sequence thereof was the sequence of SEQ ID NO. 1).

### (ii) Construction of Plasmid V-AT2

The nucleic acid sequence encoding tyrosinase derived from the genome of Ralstonia pseudosolanacearum, the sequence of SEQ ID NO. 2 (the amino acid sequence encoded thereby is the sequence of SEQ ID NO. 5), was obtained from the National Center for Biotechnology Information database.

For the foregoing nucleic acid sequence encoding tyrosinase (the sequence of SEQ ID NO. 2), primers for polymerase chain reaction (as shown in the following Table 2) and restriction enzyme cutting sites were designed to meet the requirements of multiple cloning sites of plasmid.

TABLE 2
Primers for polymerase chain reaction
designed for the foregoing nucleic
acid sequence encoding tyrosinase
(the sequence of SEQ ID NO. 2)

| Primers | Sequence | SEQ ID NO. |
|---|---|---|
| Forward | TAGAAATAATTTTGTTTAACTTTAAGAA | SEQ ID |
| primer | GGAGATATACATATGGTAGTCCGTCGTA | NO. 9 |
| | CCGT | |
| Reverse | CCAACTCAGCTTCCTTTCGGGCTTTGTT | SEQ ID |
| primer | AGCAGCCGGATCTTAAATCACTGCCACC | NO. 10 |
| | TCAA | |

The foregoing nucleic acid sequence encoding tyrosinase (the sequence of SEQ ID NO. 2) was amplified through a polymerase chain reaction using the above-mentioned primers, and then the amplified DNA fragment was purified by electrophoresis. The obtained purified DNA fragment was ligated to plasmid pZE21-MCS-1 via specific restriction enzymes to obtain plasmid V-AT2.

The map of plasmid V-AT1 is shown in FIG. 1B, which carries the nucleic acid sequence encoding tyrosinase (the sequence thereof was the sequence of SEQ ID NO. 2).

(iii) Construction of Plasmid V-AT3

The nucleic acid sequence encoding a non-secreted L-amino acid ligase derived from the genome of Serratia symbiotica, the sequence of SEQ ID NO. 3 (the amino acid sequence encoded thereby is the sequence of SEQ ID NO. 6), was obtained from the National Center for Biotechnology Information database.

For the foregoing nucleic acid sequence encoding a non-secreted L-amino acid ligase (the sequence of SEQ ID NO. 3), primers for polymerase chain reaction (as shown in the following Table 3) and restriction enzyme cutting sites were designed to meet the requirements of multiple cloning sites of plasmid.

Primers for polymerase chain reaction designed for the foregoing nucleic acid sequence encoding a non-secreted L-amino acid ligase (the sequence of SEQ ID NO. 3)

| Primer | Sequence | SEQ ID NO. |
|---|---|---|
| Forward | TAGAAATAATTTTGTTTAACTTTAAGAA | SEQ ID NO. 11 |
| primer | GGAGATATACATATGTCTGTTATCGCGG | |
| | TTCT | |
| Reverse | CTTTGTTAGCAGCCGGATCTCAGTGGTG | SEQ ID NO. 12 |
| primer | GTGGTGGTGGTGCTCGAGAGACAGCGC | |
| | GCTCT | |

The foregoing nucleic acid sequence encoding a non-secreted L-amino acid ligase (the sequence of SEQ ID NO. 3) was amplified through a polymerase chain reaction using the above-mentioned primers, and then the amplified DNA fragment was purified by electrophoresis. The obtained purified DNA fragment was ligated to plasmid pET-26b(+) via specific restriction enzymes to obtain plasmid V-AT3.

The map of plasmid V-AT3 is shown in FIG. 1C, which carries the nucleic acid sequence encoding a non-secreted L-amino acid ligase (the sequence thereof was the sequence of SEQ ID NO. 3).

(2) Construction of Genetically Modified Strains of Escherichia coli, JMD01, JMD02, JMD03 and JMD04

The plasmid V-AT1, plasmid V-AT2 and/or plasmid V-AT3 obtained above was/were introduced into competent cells of Escherichia coli BL-21, and the antibiotic(s) corresponding to the plasmid(s) was/were used for tolerance screening. After the obtained strain was confirmed that whether the length of the gene fragment which has been introduced matches the length of the gene fragment to be introduced through a polymerase chain reaction, gene sequencing was performed to confirm that the gene fragment to be introduced has indeed been introduced into Escherichia coli BL-21.

Genetically modified strains of Escherichia coli, JMD01, JMD02, JMD03 and JMD04, were constructed through the method mentioned above. Genetically modified strain of Escherichia coli, JMD01, only contains plasmid V-AT3, genetically modified strain of Escherichia coli, JMD02, only contains plasmid V-AT1, genetically modified strain of Escherichia coli, JMD03, only contains plasmid V-AT2 while genetically modified strain of Escherichia coli, JMD04, contains plasmid V-AT1 and plasmid V-AT2 at the same time.

Subsequently, dipeptide synthesis ability of the foregoing genetically modified strains of Escherichia coli, JMD01 and JMD02 were evaluated, and effect of dipeptide synthesis on melanin production was evaluated through the genetically modified strains of Escherichia coli, JMD03 and JMD04.

### 2. Medium Formula

Medium A: Lysogeny broth (Luria Bertani broth, LB) medium (which is a nutritional medium commonly used for microbial culture).

Medium B: Glucose 10 g/L, yeast extract (YE) 5 g/L, phosphate buffer (PB), and addition of antibiotic, ampicillin (100 mg/mL) or spectinomycin (40 mg/mL) or addition of both two antibiotics at the same time according to the difference of test strains.

### 3. Quantification of Alanine, Tyrosine and Dipeptide in Medium

### (1) Establishment of Respective Standard Curves for Alanine, Tyrosine and Dipeptide

Quantification of alanine, tyrosine and dipeptide was performed by high performance liquid chromatography (HPLC).

Conditions for high performance liquid chromatography are described as follows:
(i) Column: InertSustain C18 analytical column (5 μm, 4.6× 250 mm) (GL Sciences);
(ii) Standards: L-alanine standard, L-tyrosine standard and L-alanyl-L-tyrosine standard (TCI, A1263) respectively were dissolved in water;
(iii) Mobile phase: 5 mM sodium 1-octanesulfonate, 10 mM KH₂PO₄ and 0.2% H₃PO₄ in H₂O/methanol=50/50;
(iv) Flow rate: 0.6 mL/minute;
(v) Temperature: 26° C.;
(vi) Detector: UV detector;
(vii) UV wavelength: 203 nm.

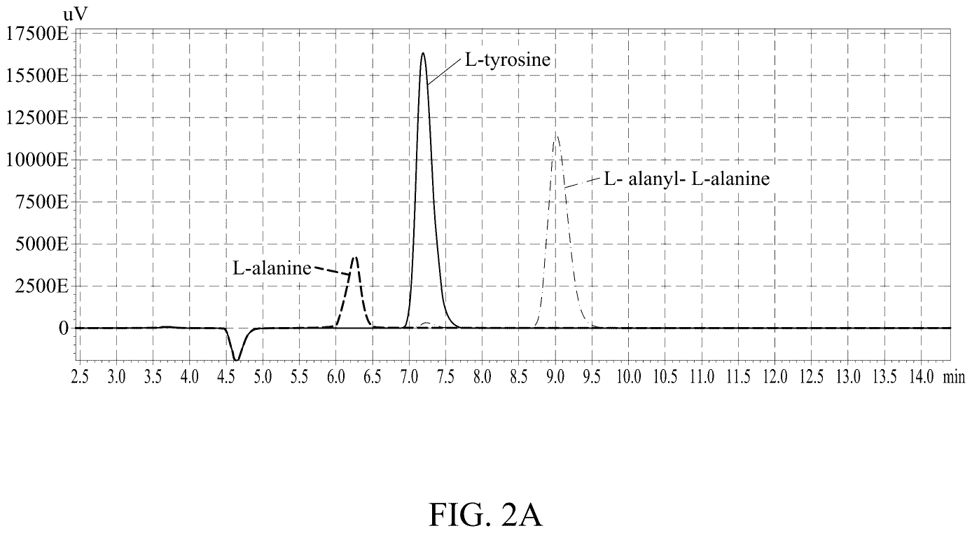

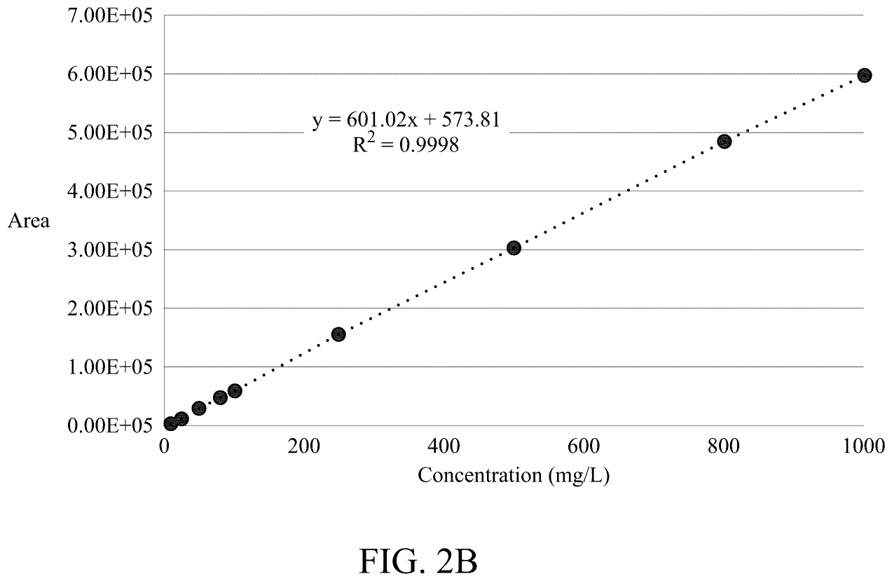

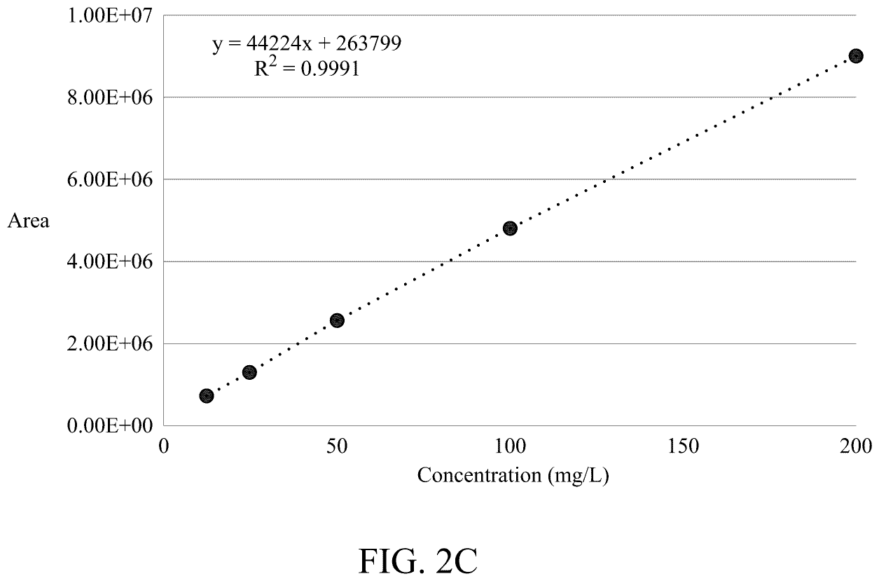

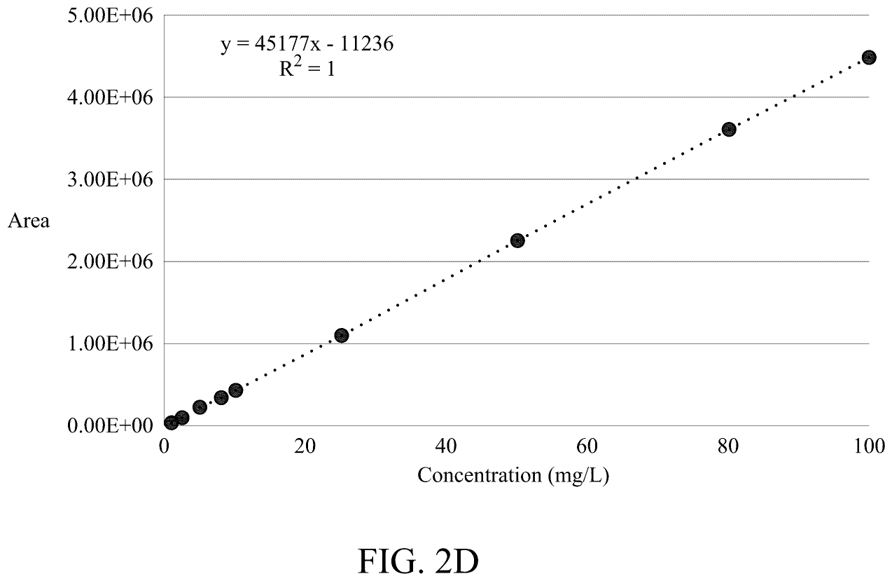

L-alanine standard (1.0 g/L), L-tyrosine standard (0.5 g/L) and L-alanyl-L-tyrosine standard (0.5 g/L) were analyzed through high performance liquid chromatography under the foregoing conditions. The results are shown in FIG. 2A.

Moreover, through the above analysis conditions, a standard curve of the concentration of L-alanine versus the peak area of L-alanine in high-performance liquid chromatography (FIG. 2B), a standard curve of the concentration of L-tyrosine versus the peak area of L-tyrosine in high-performance liquid chromatography (FIG. 2C), and a standard curve of the concentration of dipeptide (L-alanyl-L-tyrosine) versus the peak area of dipeptide in high-performance liquid chromatography (FIG. 2D) were established. Standard curve ranges: L-alanine: 10-1000 mg/L; L-tyrosine: 12.5-200 mg/L; dipeptide (L-alanyl-L-tyrosine): 1-100 mg/L.

### (2) Determination for Contents of Alanine, Tyrosine and Dipeptide in Medium

Medium was subjected to high-performance liquid chromatography under the foregoing conditions to respectively obtain the peak areas of alanine, tyrosine and dipeptide, and then the obtained peak areas of alanine, tyrosine and dipeptide were respectively used to calculate the contents of alanine, tyrosine and dipeptide in medium according to the above-mentioned standard curves respectively corresponding to alanine, tyrosine and dipeptide.

### 4. Analysis Method for Melanin Production of Bacterial Strains

Quantitative analysis for melanin production was based on the following literatures: (1) Yuksel Keleş et al; Extraction, purification, antioxidant properties and stability conditions of phytomelanin pigment on the sunflower seeds (DOI: 10.21448/ijsm.377470); and (2) Microbial Production of Melanin Pigments from Caffeic Acid and L-Tyrosine Using Streptomyces glaucescens and FCS-ECH-Expressing Escherichia coli. (DOI: 10.3390/ijms22052413).

### (1) Establishment of Standard Curve

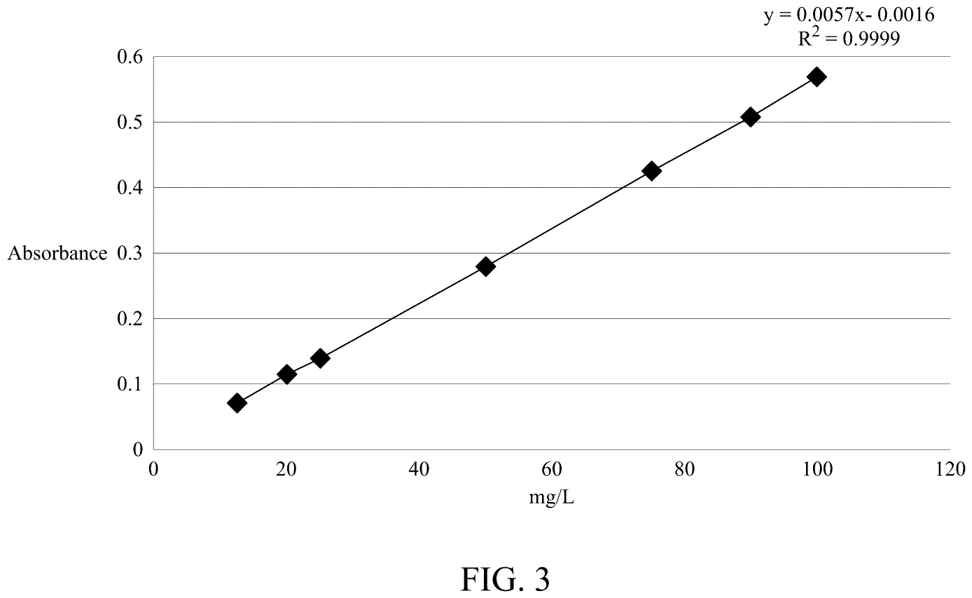

Melanin standard was prepared to standard samples with different concentrations using 0.1N NaOH. Absorbance values (OD₄₀₀) of standard samples with different concentrations were measured at a wavelength of 400 nm by a spectrophotometer (Table 4), and a standard curve of melanin concentration versus OD₄₀₀ was established (FIG. 3).

TABLE 4
Absorbance values measured at wavelength 400
nm for various concentrations of melanin

Absorbance value of blank (NaOH)
0.046

| | | Absorbance value |
|---|---|---|
| | | (absorbance value of the |
| | Absorbance | blank group has been |
| Melanin (mg/L) | value | deducted) |
| 200 | 1.187 | 1.141 |
| 100 | 0.614 | 0.568 |
| 90 | 0.552 | 0.506 |
| 75 | 0.470 | 0.424 |
| 50 | 0.324 | 0.278 |
| 25 | 0.185 | 0.139 |
| 20 | 0.159 | 0.113 |
| 12.5 | 0.117 | 0.071 |
| 6.25 | 0.082 | 0.036 |

### (2) Determination of Melanin Production of Bacterial Strains

After the culture was completed, the bacterial suspension was centrifuged, and then the supernatant was removed. 0.1 N NaOH was added to the bacterial cells, and then cell disruption was conducted by ultrasonic vibration to obtain a cell lysate solution.

Next, the cell lysate solution was centrifuged, and the absorbance value of the obtained supernatant was measured at a wavelength of 400 nm. After that, the measured absorbance value was used to calculate the melanin content in the supernatant according to the standard curve of melanin concentration versus OD 400 mentioned above to confirm the melanin production of the bacteria.

### B. Experiments

### Example 1

Confirmation that L-Amino Acid Ligase is Secreted to the Outside of Bacterial Strain

The modified strains of Escherichia coli, JMD01 and JMD02, were separately inoculated into separate 14 mL culture tubes containing 2 mL of Medium A, and cultured overnight at 37° C. to activate the bacterial strains.

After that, the modified strains of Escherichia coli, JMD01 and JMD02, which had been activated were separately inoculated into separate shake flasks containing Medium B, and cultured at 37° C., pH 7.0 until the OD 600 values thereof were 0.4-0.6 (about 6 hours). Next, the culture temperature was changed to 30° C., 0.5 mM isopropyl β-D-1-thiogalactopyranoside (IPTG) was added as an inducer to the foregoing shake flasks, and the culture for the modified strains was continued for 24 hours.

Afterwards, the obtained bacterial suspension was centrifuged to obtain the supernatant for sodium dodecyl sulfate polyacrylamide gel electrophoresis (SDS-PAGE). The detailed steps of sodium dodecyl sulfate polyacrylamide gel electrophoresis are described as follows.

1 mL of bacterial suspension was centrifuged at 12,000 rpm for 10 minutes to obtain a supernatant, and the obtained supernatant was added to a 4-fold concentrated sample buffer to form a sample.

The sample was heated at 95° C. for 25 minutes to complete a protein pretreatment. Then, the sample to which the protein pretreatment had been completed was subjected to sodium dodecyl sulfate polyacrylamide gel electrophoresis. Conditions for sodium dodecyl sulfate polyacrylamide gel electrophoresis are described as follows:
(1) Electrophoresis gel: Mini-PROTEAN TGX Precast Gels;
(2) Electrophoresis conditions: 60 volts for 20 minutes, followed by 110 volts for 60 minutes;

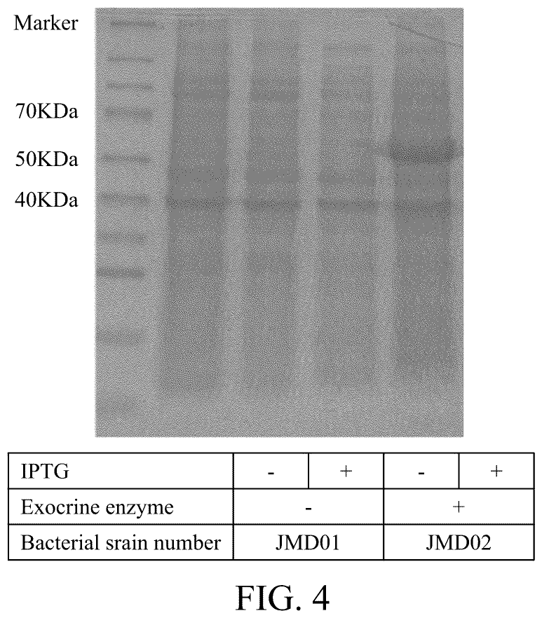

After electrophoresis, the gel was stained with Coomassie Stain (Bio-Safe; BIO-RAD) for analysis. The results are shown in FIG. 4.

Based on FIG. 4, it is known that for the modified strain of Escherichia coli, JMD01, no matter whether the inducer IPTG is added or not, the signal of L-amino acid ligase cannot be detected in the supernatant of the bacterial suspension (the cultured medium). In contrast, under the condition that IPTG was added to the modified strain of Escherichia coli, JMD02, the supernatant of the bacterial suspension (the cultured medium) showed a significant signal at the 50 kDa position on the electrophoresis gel, representing that the L-amino acid ligase expressed from the plasmid carrying a secreted signal can accumulate in the culture medium, and the signal can be displayed through sodium dodecyl sulfate polyacrylamide gel electrophoresis.

Specifically, the modified strain of Escherichia coli, JMD01, cannot secrete L-amino acid ligase to the outside of cell while the modified strain of Escherichia coli, JMD02, can secrete L-amino acid ligase to the outside of cell.

### Example 2

### Confirmation of Tyrosine Concentration in Cultured Medium Reduced by L-Amino Acid Ligase Secreted to the Outside of Cell

The modified strains of Escherichia coli, JMD01 and JMD02, were separately inoculated into separate 14 mL culture tubes containing 2 mL of Medium A, and cultured overnight at 37° C. to activate the bacterial strains.

After that, the modified strains of Escherichia coli, JMD01 and JMD02, which had been activated were separately inoculated into separate shake flasks containing Medium B, 5 g/L alanine and 5 g/L tyrosine, and cultured at 37° C., pH 7.0 until the OD₆₀₀ values thereof were 0.4-0.6 (about 6 hours). Next, 0.5 mM isopropyl β-D-1-thiogalactopyranoside (IPTG) was added as an inducer to the foregoing shake flasks, the culture temperature was changed to 30° C., and the culture for the modified strains was continued for 40 hours, wherein the bacterial suspension was sampled at 0, 8, 24, 32 and 40 hours of culture.

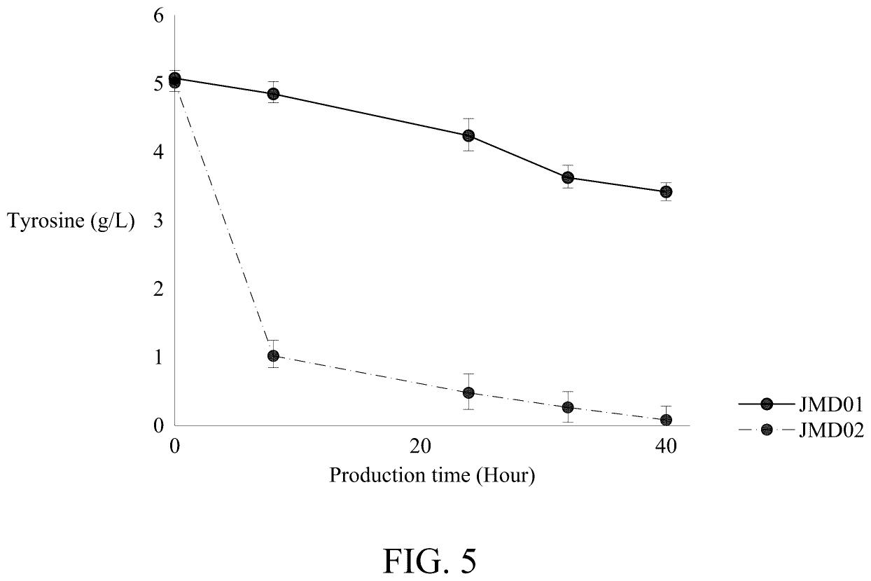

Afterwards, the obtained bacterial suspension was centrifuged to obtain the supernatant. The concentration of tyrosine in the supernatant (the cultured medium) of the bacterial suspension was confirmed by high performance liquid chromatography. The results are shown in FIG. 5.

According to FIG. 5, it is known that since there is no signal for L-amino acid ligase secreted to the outside of cell in the supernatant (the cultured medium) of the bacterial suspension of the modified strain of Escherichia coli, JMD01 (refer to FIG. 4), the concentration of remaining tyrosine is relatively high. In contrast, the supernatant (the cultured medium) of the bacterial suspension of the modified strain of Escherichia coli, JMD02, contains the signal for L-amino acid ligase secreted to the outside of cell (refer to FIG. 4), and the concentration of remaining tyrosine decreases with time, representing that the L-amino acid ligase secreted to the outside of cell can effectively utilize the tyrosine in the medium.

Specifically, compared to the modified strain of Escherichia coli, JMD01, the modified strain of Escherichia coli, JMD02 has better tyrosine utilization efficiency through the L-amino acid ligase secreted to the outside of cell.

### Example 3

### Confirmation of Dipeptide Concentration in Cultured Medium Increased by L-Amino Acid Ligase Secreted to the Outside of Cell

The modified strains of Escherichia coli, JMD01 and JMD02, were separately inoculated into separate 14 mL culture tubes containing 2 mL of Medium A, and cultured overnight at 37° C. to activate the bacterial strains.

After that, the modified strains of Escherichia coli, JMD01 and JMD02, which had been activated were separately inoculated into separate shake flasks containing Medium B, 10 g/L alanine and 10 g/L tyrosine, and cultured at 37° C., pH 7.0 until the OD₆₀₀ values thereof were 0.4-0.6 (about 6 hours). Next, 0.5 mM isopropyl β-D-1-thiogalactopyranoside (IPTG) was added as an inducer to the foregoing shake flasks, the culture temperature was changed to 30° C., and the culture for the modified strains was continued for 40 hours.

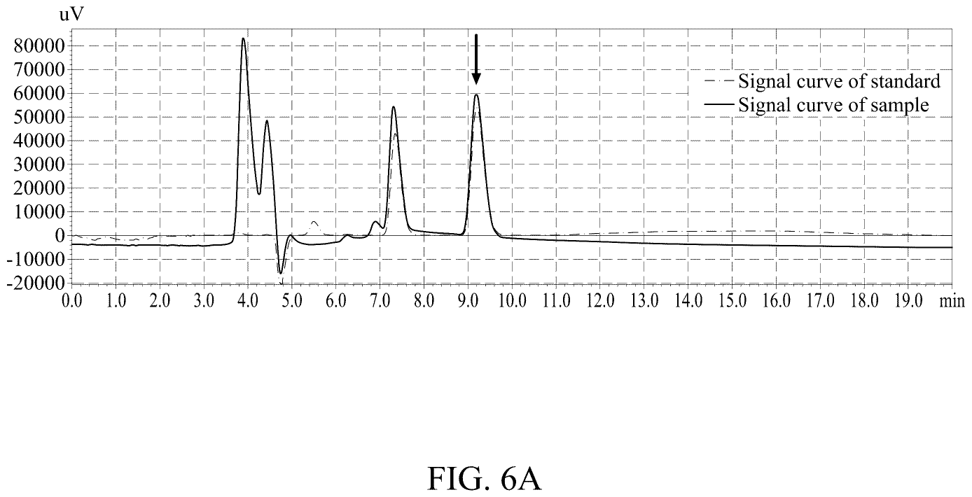

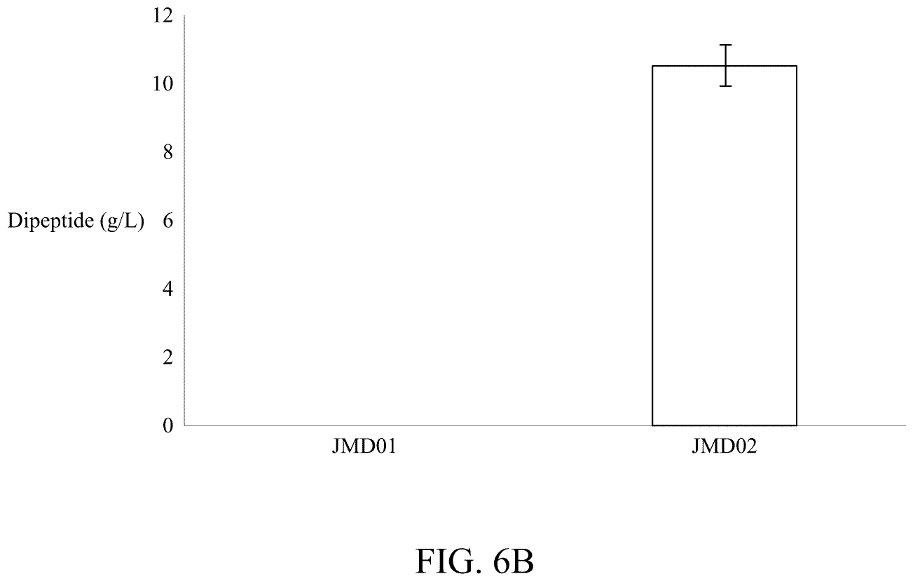

Afterwards, the obtained bacterial suspension was centrifuged to obtain the supernatant. The concentration of dipeptide in the supernatant (the cultured medium) of the bacterial suspension was confirmed by high performance liquid chromatography. The results are shown in FIG. 6A and FIG. 6B.

FIG. 6A shows the results of high-performance liquid chromatography for the supernatant (the cultured medium) of the bacterial suspension of the modified strain of Escherichia coli, JMD02. The dotted line is the signal curve of the standard while the solid line is the signal curve of the supernatant (the cultured medium) (the sample) of the bacterial suspension of the modified strain of Escherichia coli, JMD02. The peak at 9.2 minutes in the signal curve of the standard is the signal of the dipeptide while the sample has an overlapping signal at the same position. No signal of the dipeptide was detected in the supernatant (the cultured medium) of the bacterial suspension of the modified strain of Escherichia coli, JMD01, by high performance liquid chromatography. In addition, FIG. 6B shows results of respectively calculating the concentrations of dipeptide in the supernatants (the cultured media) of the bacterial suspensions of the modified strains of Escherichia coli, JMD01 and JMD02, based on the previously obtained standard curve of the concentration of dipeptide (L-alanyl-L-tyrosine) versus the peak area of dipeptide in high performance liquid chromatography.

According to FIG. 6A and FIG. 6B, it is known that the modified strain of Escherichia coli, JMD02 can significantly increase the concentration of dipeptide in the cultured medium, and in contrast, no dipeptide is produced in the cultured medium of the modified strain of Escherichia coli, JMD01.

### Example 4

### Confirmation of the Ability of Secreted L-Amino Acid Ligase to Produce Dipeptide

The modified strain of Escherichia coli, JMD02, was inoculated into a 14 mL culture tube containing 2 mL of Medium A, and cultured overnight at 37° C. to activate the bacterial strain.

After that, the modified strain of Escherichia coli, JMD02, which had been activated was inoculated into a shake flask containing Medium B, 10 g/L tyrosine and different concentrations (1 g/L, 5 g/L or 10 g/L) of alanine, and cultured at 37° C., pH 7.0 until the OD₆₀₀ value thereof was 0.4-0.6 (about 6 hours). Next, 0.5 mM isopropyl β-D-1-thiogalactopyranoside (IPTG) was added as an inducer to the foregoing shake flask, the culture temperature was changed to 30° C., and the culture for the modified strains was continued for 40 hours, wherein the bacterial suspension was sampled at 0, 8, 24, 32 and 40 hours of culture.

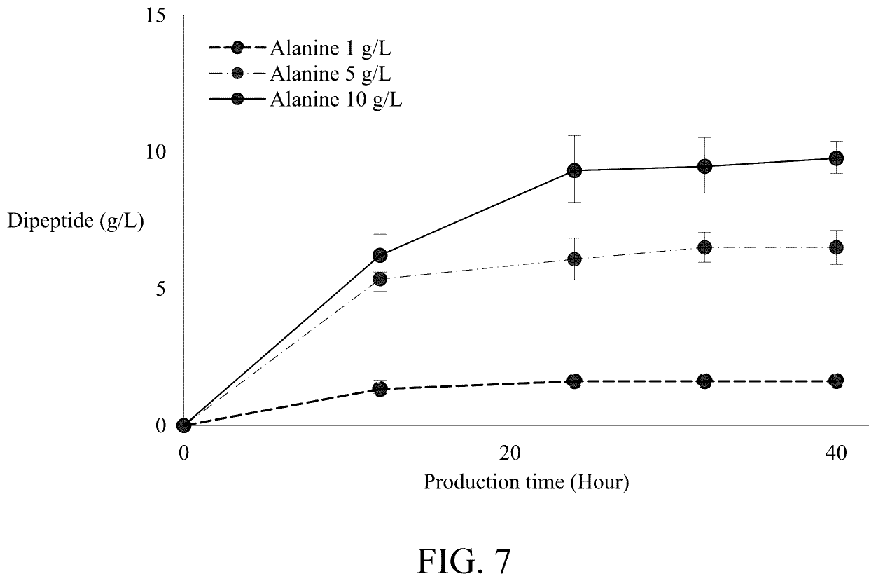

Afterwards, the obtained bacterial suspension was centrifuged to obtain the supernatant. The concentration of dipeptide in the supernatant (the cultured medium) of the bacterial suspension was confirmed by high performance liquid chromatography. The results are shown in FIG. 7.

Based on FIG. 7, it is known that as the concentration of alanine increases, the concentration of dipeptide produced by the modified strain of Escherichia coli, JMD02, also increases, and reaches the highest concentration, 9.82 g/L, at 40 hours of production.

### Example 5

### Confirmation of Producing Melanin Using Dipeptide

The modified strain of Escherichia coli, JMD02, was inoculated into a 14 mL culture tube containing 2 mL of Medium A, and cultured overnight at 37° C. to activate the bacterial strain.

After that, the modified strain of Escherichia coli, JMD02, which had been activated was inoculated into a shake flask containing Medium B, 10 g/L alanine and 10 g/L tyrosine and cultured at 37° C., pH 7.0 until the OD₆₀₀ value thereof was 0.4-0.6 (about 6 hours). Next, 0.5 mM isopropyl β-D-1-thiogalactopyranoside (IPTG) was added as an inducer to the foregoing shake flask, the culture temperature was changed to 30° C., and the culture for the modified strain was continued for 40 hours.

Afterwards, the obtained bacterial suspension was centrifuged to obtain the supernatant, and the concentration of dipeptide in the supernatant (the cultured medium) of the bacterial suspension was confirmed by high performance liquid chromatography. Then, the concentration of the dipeptide in the supernatant of the bacterial suspension (the cultured medium) was adjusted to 10 g/L or 5 g/L, and 10 g/L glucose and 5 g/L yeast extract were added to the bacterial suspension to serve as a medium for subsequent culture for the modified strain of Escherichia coli, JMD03.

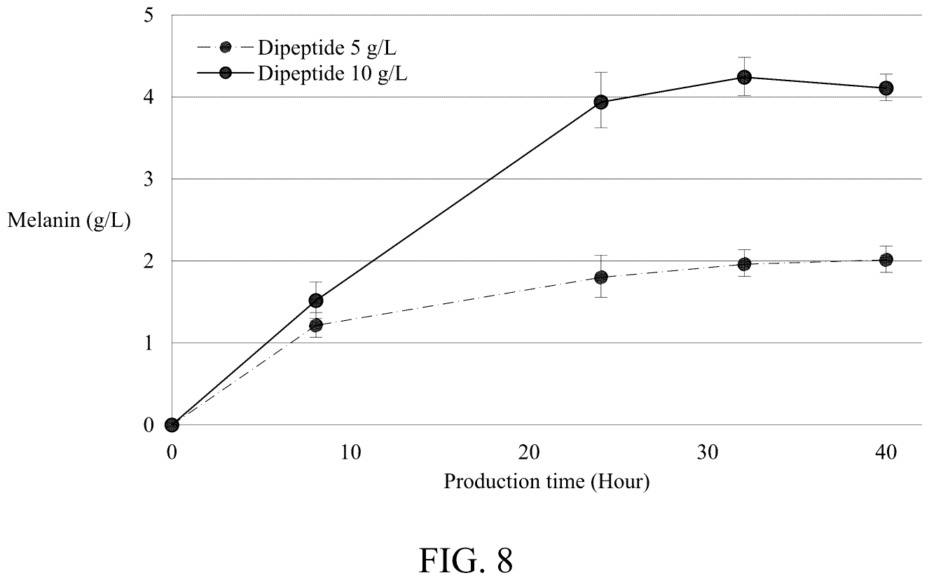

The modified strain of Escherichia coli, JMD03, which had been activated was inoculated into this medium and cultured at 37° C., pH 7.0. After culturing for 4 hours, 0.5 mM isopropyl β-D-1-thiogalactopyranoside (IPTG) as an inducer and 20 mg/L copper sulfate were added, and the culture temperature was changed to 30° C. to culture. The bacterial suspension was sampled at different time points and centrifuged at 12,000 rpm for 10 minutes to collect the bacterial cells for performing melanin concentration analysis. The results are shown in FIG. 8.

According to FIG. 8, it is known that the modified strain of Escherichia coli, JMD03, can synthesize melanin through the dipeptide in the supernatant of the bacterial suspension of the modified strain of Escherichia coli, JMD02, while the melanin production increases with time and the concentration of dipeptide.

### Example 6

Comparison of Melanin Production Between Modified Strains of Escherichia coli, JMD03 and JMD04

The modified strains of Escherichia coli, JMD03 and JMD04, were separately inoculated into separate 14 mL culture tubes containing 2 mL of Medium A, and cultured overnight at 37° C. to activate the bacterial strains.

After that, the modified strains of Escherichia coli, JMD03 and JMD04, which had been activated were separately inoculated into separate shake flasks containing Medium B, 25 g/L alanine and 10 g/L tyrosine, and cultured at 37° C., pH 7.0 until the OD₆₀₀ values thereof were 0.4-0.6 (about 6 hours). Next, 0.5 mM isopropyl β-D-1-thiogalactopyranoside (IPTG) as an inducer and 20 mg/L copper sulfate were added to the foregoing shake flasks, the culture temperature was changed to 30° C., and the culture for the modified strains was continued for 40 hours.

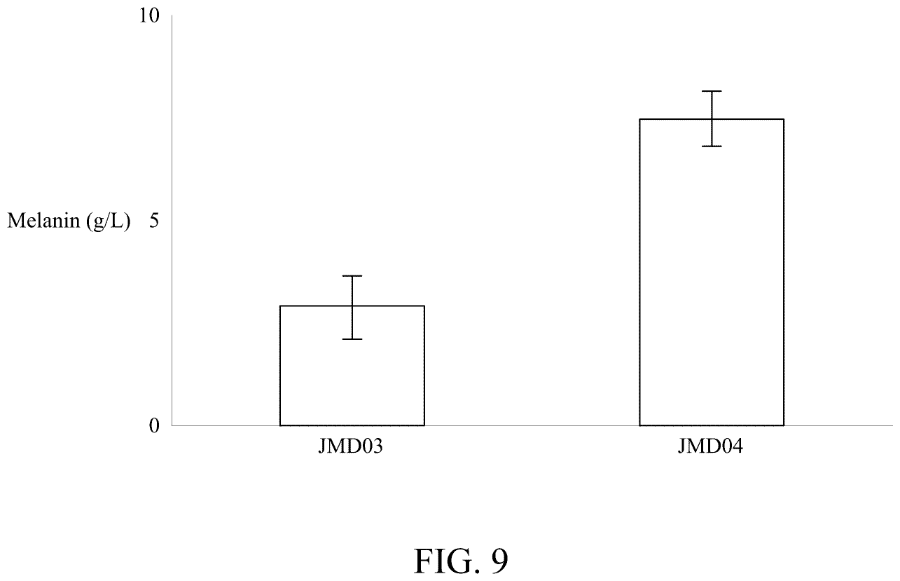

Next, the bacterial suspension was centrifuged at 12,000 rpm for 10 minutes to collect the bacterial cells for performing melanin concentration analysis. The results are shown in FIG. 9.

Based on FIG. 9, it is known that compared to the modified strain of Escherichia coli, JMD03, without secreted L-amino acid ligase, the modified strain of Escherichia coli, JMD04, with secreted L-amino acid ligase shows significantly higher melanin production, representing that secreted L-amino acid ligase can effectively increase melanin production.

### Example 7

Effects of Different Tyrosine Addition Amounts on Melanin Production of Modified Strain of Escherichia coli, JMD04

The modified strain of Escherichia coli, JMD04, was inoculated into a 14 mL culture tube containing 2 mL of Medium A, and cultured overnight at 37° C. to activate the bacterial strain.

After that, the modified strain of Escherichia coli, JMD04, which had been activated was inoculated into a shake flask containing Medium B, 25 g/L alanine and different concentrations (1 g/L, 5 g/L, 10 g/L or 25 g/L) of tyrosine and cultured at 37° C., pH 7.0 until the OD₆₀₀ value thereof was 0.4-0.6 (about 6 hours). Next, 0.5 mM isopropyl β-D-1-thiogalactopyranoside (IPTG) as an inducer and 20 mg/L copper sulfate were added to the foregoing shake flask, the culture temperature was changed to 30° C., and the culture for the modified strains was continued for 40 hours.

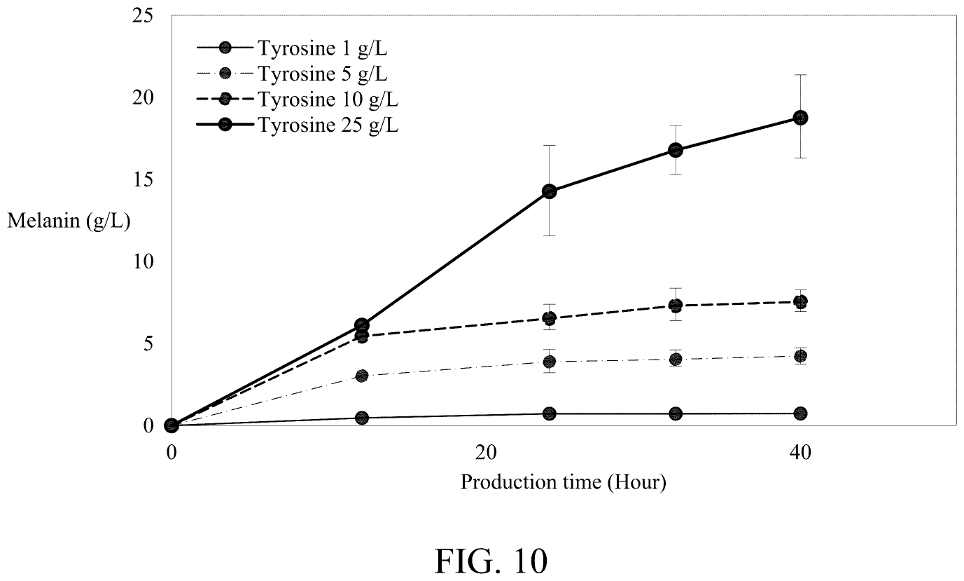

Next, the bacterial suspension was centrifuged at 12,000 rpm for 10 minutes to collect the bacterial cells for performing melanin concentration analysis. The results are shown in FIG. 10.

According to FIG. 10, it is known that the melanin production of the modified strain of Escherichia coli, JMD04, increases with the increase of tyrosine concentration.

In addition, the modified strain of Escherichia coli, JMD04, was stored at −80° C. at a ratio of glycerol to bacterial suspension of 1:1, and was deposited in the German Collection of Microorganisms and Cell Cultures GmbH (DSMZ) on Nov. 4, 2024, with the deposit number of DSM 35223, and also deposited in Bioresource Collection and Research Centre (BCRC) of Food Industry Research and Development Institute (FIRDI) (ROC.) on Nov. 5, 2024, with the deposit number of BCRC 940702.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A genetically modified microorganism for enhancing melanin production efficiency, comprising:
a first exogenous nucleic acid, comprising a nucleic acid encoding a secreted L-amino acid ligase, wherein the secreted L-amino acid ligase is capable of linking two amino acids to form a dipeptide; and
a second exogenous nucleic acid, comprising a nucleic acid encoding tyrosinase.

2. The genetically modified microorganism for enhancing melanin production efficiency as claimed in claim 1, wherein a source microorganism of the genetically modified microorganism for enhancing melanin production efficiency comprises a bacterium, an actinomycete, a yeast or a mold, wherein the bacterium comprises Escherichia coli, Corynebacterium glutamicum or Bacillus subtilis, the actinomycete comprises Streptomyces lividans, Streptomyces violaceoruber or Streptomyces coelicolor, the yeast comprises Yarrowia lipolytica, Saccharomyces cerevisiae or Pichia pastoris, and the mold comprises Aspergillus oryzae or Aspergillus terreus.

3. The genetically modified microorganism for enhancing melanin production efficiency as claimed in claim 2, wherein the bacterium is Escherichia coli.

4. The genetically modified microorganism for enhancing melanin production efficiency as claimed in claim 1, wherein the sequence of the nucleic acid encoding a secreted L-amino acid ligase comprises the sequence of SEQ ID NO. 1.

5. The genetically modified microorganism for enhancing melanin production efficiency as claimed in claim 1, wherein the sequence of the nucleic acid encoding a secreted L-amino acid ligase is derived from Serratia symbiotica, wherein the sequence of the nucleic acid encoding a secreted L-amino acid ligase comprises the sequence of SEQ ID NO. 1.

6. The genetically modified microorganism for enhancing melanin production efficiency as claimed in claim 1, wherein the dipeptide comprises a first amino acid and a second amino acid, and the C-terminal of the first amino acid is linked to the N-terminal of the second amino acid, and the second amino acid is tyrosine.

7. The genetically modified microorganism for enhancing melanin production efficiency as claimed in claim 6, wherein the first amino acid is alanine.

8. The genetically modified microorganism for enhancing melanin production efficiency as claimed in claim 1, wherein the sequence of the nucleic acid encoding tyrosinase comprises the sequence of SEQ ID NO. 2.

9. The genetically modified microorganism for enhancing melanin production efficiency as claimed in claim 1, wherein the sequence of the nucleic acid encoding tyrosinase is derived from Ralstonia pseudosolanacearum, wherein the sequence of the nucleic acid encoding tyrosinase comprises the sequence of SEQ ID NO. 2.

10. The genetically modified microorganism for enhancing melanin production efficiency as claimed in claim 1, wherein the genetically modified microorganism for enhancing melanin production efficiency is a genetically modified strain of Escherichia coli with the deposit number of DSM 35223.

11. A method for producing melanin, comprising:
(a) culturing the genetically modified microorganism for enhancing melanin production efficiency as claimed in claim 1 in the presence of a first amino acid and a second amino acid, in the presence of a dipeptide, or in the presence of a first amino acid, a second amino acid and a dipeptide,
wherein the second amino acid is tyrosine; and
wherein the dipeptide comprises a third amino acid and a fourth amino acid, and the C-terminal of the third amino acid is linked to the N-terminal of the fourth amino acid, and the third amino acid is the same as the first amino acid while the fourth amino acid is the same as the second amino acid.

12. The method for producing melanin as claimed in claim 11, wherein under the condition that it is in the presence of a first amino acid and a second amino acid, the concentration of the first amino acid is 0.1-50 g/L, and the first amino acid is alanine.

13. The method for producing melanin as claimed in claim 11, wherein under the condition that it is in the presence of a first amino acid and a second amino acid, the concentration of the second amino acid is 0.1-50 g/L, and the first amino acid is alanine.

14. The method for producing melanin as claimed in claim 11, wherein under the condition that it is in the presence of a dipeptide, the concentration of the dipeptide is 0.1-50 g/L, and the third amino acid is alanine.

15. The method for producing melanin as claimed in claim 11, wherein under the condition that it is in the presence of a first amino acid, a second amino acid and a dipeptide, the concentrations of the first amino acid, the second amino acid and the dipeptide independently are 0.05-25 g/L, and the first amino acid is alanine.

16. The method for producing melanin as claimed in claim 11, wherein a source microorganism of the genetically modified microorganism for enhancing melanin production efficiency comprises a bacterium, and the bacterium is Escherichia coli.

17. The method for producing melanin as claimed in claim 11, wherein the sequence of the nucleic acid encoding a secreted L-amino acid ligase is derived from Serratia symbiotica, wherein the sequence of the nucleic acid encoding a secreted L-amino acid ligase comprises the sequence of SEQ ID NO. 1.

18. The method for producing melanin as claimed in claim 11, wherein the sequence of the nucleic acid encoding tyrosinase is derived from Ralstonia pseudosolanacearum, wherein the sequence of the nucleic acid encoding tyrosinase comprises the sequence of SEQ ID NO. 2.

19. The method for producing melanin as claimed in claim 11, wherein the genetically modified microorganism for enhancing melanin production efficiency is cultured at 25-40° C. and pH 6.0-8.0 for 4-50 hours.

20. The method for producing melanin as claimed in claim 11, further comprising (b) extracting melanin from the genetically modified microorganism for enhancing melanin production efficiency after completing the step (a).

21. The method for producing melanin as claimed in claim 11, wherein the genetically modified microorganism for enhancing melanin production efficiency is a genetically modified strain of Escherichia coli with the deposit number of DSM 35223.
